SEQUENCE LISTING (10 sequences)

SEQ ID NO: 1 - DNA, 19 bp, synthetic construct
  source 1..19: mol_type = other DNA
gtgccagcmgccgcggtaa

SEQ ID NO: 2 - DNA, 20 bp, synthetic construct
  source 1..20: mol_type = other DNA
ggactachvgggtwtctaat

SEQ ID NO: 3 - DNA, 17 bp, synthetic construct
  source 1..17: mol_type = other DNA
acggccgcaaggttaaa

SEQ ID NO: 4 - DNA, 23 bp, synthetic construct
  source 1..23: mol_type = other DNA
gctggatgtcaagagtaggtaag

SEQ ID NO: 5 - DNA, 24 bp, synthetic construct
  source 1..24: mol_type = other DNA
acaagcggtggagcatgtggttta

SEQ ID NO: 6 - DNA, 19 bp, synthetic construct
  source 1..19: mol_type = other DNA
cggaatcactgggcgtaaa

SEQ ID NO: 7 - DNA, 22 bp, synthetic construct
  source 1..22: mol_type = other DNA
gttccaccaacctctaccatac

SEQ ID NO: 8 - DNA, 19 bp, synthetic construct
  source 1..19: mol_type = other DNA
cggaatcactgggcgtaaa

SEQ ID NO: 9 - DNA, 17 bp, synthetic construct
  source 1..17: mol_type = other DNA
cctaygggrbgcascag

SEQ ID NO: 10 - DNA, 20 bp, synthetic construct
  source 1..20: mol_type = other DNA
ggactacnngggtatctaat

(19) United States
(12) Patent Application Publication
(10) Pub. No.: US 2026/0347757 A1
(43) Pub. Date: Nov. 5, 2026

(54) BACTERIAL COMPOSITIONS AND METHODS FOR PROMOTING PLANT GROWTH

(71) Applicant: University of Tasmania, Tasmania (AU)

(72) Inventors: Calum Rae Wilson, Tasmania (AU); David Scott Nichols, Tasmania (AU); Eda Marie Barsolate-Wei, Tasmania (AU)

(73) Assignee: University of Tasmania, Tasmania (AU)

(21) Appl. No.: 18/880,910

(22) PCT Filed: Jul. 5, 2023
(86) PCT No.: PCT/AU2023/050619

§ 371 Date: Jan. 3, 2025

(30) Foreign Application Priority Data
Jul. 5, 2022 (AU) 2022901887

(51) Int. Cl. C12N 1/205; A01N 63/20; A01P 21/00; C12R 1/01

(52) U.S. Cl. CPC C12N 1/205; A01N 63/20; A01P 21/00; C12R 2001/01

(57) ABSTRACT

The present invention relates to bacterial strains and compositions, and their use for controlling diseases in plants and for promoting plant growth. In one aspect, the isolated bacterial strain RR15 of the genus Pantoea, deposited under Accession Number V22/011015 on 9 Jun. 2022 with the National Measurement Institute, Australia, is provided. Methods for promoting plant growth, promoting plant tuber growth, inhibiting plant root infection by a pathogen, and inhibiting plant root disease by a pathogen, using the bacterial strains and compositions are also provided

### PRIORITY CLAIM

This application claims priority from Australian provisional patent application number 2022901887 filed on 5 Jul. 2022, the contents of which are to be taken as incorporated herein by this reference.

### FIELD OF THE INVENTION

The present invention relates generally to bacterial compositions and their use for controlling diseases in plants and for promoting plant growth.

### BACKGROUND OF THE INVENTION

A multitude of plant diseases and pests reduce crop yields, which renders agricultural food production challenging. Root exudation is an important process in the rhizosphere of plants for mediating plant-microbe interactions. For example, root exudates in the rhizosphere have a number of roles, including inducing chemotaxis, microbial growth inhibition and stimulation, as well as modulating pathogen colonisation and activation. In many cases pathogen activation by host root exudates is a common occurrence in a number of soil-borne pathosystems, particularly for environmentally resistant spore producing pathogens.

The initial stage of infection by soil-borne pathogens and beneficial interactions with microbes is root colonisation. Chemical cues released by plants and microorganisms control this dynamic connection in such a manner that plant roots commence cross talk with soil bacteria by creating signals that the microbes detect, which in turn generates signals that initiate colonisation.

In potatoes for example, powdery scab of tubers has long been regarded to be the principal disease caused by Spongospora subterranea f. sp. subterranea infection, resulting in a decline in harvested tuber quality. In this regard, potato roots produce chemical-specific stimulatory compounds involved in early germination of S. subterranea resting spores. This corroborates findings of other pathosystems whereby individual host-specific compounds found within root exudates chemotactically attract pathogen spores thereby facilitating infection. Therefore, the study of multitrophic interactions among organisms to better understand plant pathogenesis and disease management is becoming increasingly important. Indeed, multitrophic interactions are now widely regarded as mediating both the capacity of soil pathogens to produce plant disease and the ability of bacterial antagonists to suppress disease.

Rhizosphere-inhabiting microorganisms possess a variety of qualities for enhancing plant health and for priming plants against a wide range of diseases, either directly or indirectly. Plants, for example, emit root exudates that are selective for specific bacterial strains, and the latter, in turn, release metabolites that are advantageous to the plant. Accordingly, there is a need to identify rhizosphere-inhabiting microorganisms associated with plants that can be used to promote health and growth of the plant, as well as inhibit plant root infection and inhibit plant root disease mediated by soil-borne pathogens.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

The present invention is predicated, in part, on the identification of bacterial strains which have the ability to degrade pathogen stimulating compounds within plant root exudates and can promote plant development and growth.

Accordingly, in a first aspect the present invention provides an isolated bacterial strain RR15 of the genus Pantoea, deposited under Accession Number V22/011015 on 9 Jun. 2022 with the National Measurement Institute, Australia.

In some embodiments, the present invention provides a culture of the bacterial strain of the first aspect of the invention. In some embodiments, a supernatant obtained from the culture is provided. In some embodiments, a composition comprising the bacterial strain, the culture, and/or the supernatant, is provided.

In some embodiments, the bacterial strain promotes plant growth. In some embodiments, the bacterial strain promotes plant root growth. In some embodiments, the bacterial strain promotes plant tuber yield. In some embodiments, tuber size and/or weight is promoted. In some embodiments, the plant is of the Solanaceae family. In some embodiments, the plant is of the species Solanum tuberosum.

In some embodiments, the bacterial strain inhibits plant root infection by a pathogen. In some embodiments, the bacterial strain inhibits plant root disease by a pathogen. In some embodiments, the pathogen is Spongospora subterranea.

In some embodiments, the composition further comprises a biological agent. In some embodiments, the biological agent is a plant growth promoting agent. In some embodiments, the composition further comprises a bacterial stabilising agent. In some embodiments, the bacterial stabilising agent is xanthan gum.

In a further aspect, the present invention provides a method for promoting plant growth, the method comprising applying to the plant or to soil in which the plant will be, or is, propagated in, an effective amount of the bacterial strain, culture, supernatant, and/or composition, of the first aspect of the invention.

In a further aspect, the present invention provides a method for promoting plant tuber yield, the method comprising applying to the plant or to soil in which the plant will be, or is, propagated in, an effective amount of the bacterial strain, culture, supernatant, and/or composition, of the first aspect of the invention. In some embodiments, tuber size and/or weight is promoted.

In a further aspect, the present invention provides a method for inhibiting plant root infection by a pathogen, the method comprising applying to the plant or to soil in which the plant will be, or is, propagated in, an effective amount of the bacterial strain, culture, supernatant, and/or composition, of the first aspect of the invention.

In a further aspect, the present invention provides a method for inhibiting plant root disease by a pathogen, the method comprising applying to the plant or to soil in which the plant will be, or is, propagated in, an effective amount of the bacterial strain, culture, supernatant, and/or composition, of the first aspect of the invention.

In a further aspect, the present invention provides a method of promoting plant growth, of promoting plant tuber yield, of inhibiting plant root infection by a pathogen, or of inhibiting plant root disease by a pathogen, the method comprising applying to the plant or to soil in which the plant will be, or is, propagated in, an effective amount of the bacterial strain, culture, supernatant, and/or composition, of the first aspect of the invention.

In a further aspect, the present invention provides a composition comprising an isolated bacterial strain RR15 of the genus Pantoea, deposited under Accession Number V22/011015 on 9 Jun. 2022 with the National Measurement Institute, Australia.

In a further aspect, the present invention provides a composition comprising a culture of an isolated bacterial strain RR15 of the genus Pantoea, deposited under Accession Number V22/011015 on 9 Jun. 2022 with the National Measurement Institute, Australia.

In a further aspect, the present invention provides a composition comprising a supernatant of a culture of an isolated bacterial strain RR15 of the genus Pantoea, deposited under Accession Number V22/011015 on 9 Jun. 2022 with the National Measurement Institute, Australia.

In some embodiments, the bacterial strain of the composition promotes plant growth. In some embodiments, the bacterial strain promotes plant root growth. In some embodiments, the bacterial strain promotes plant tuber yield. In some embodiments, tuber size and/or weight is promoted. In some embodiments, the plant is of the Solanaceae family. In some embodiments, the plant is of the species Solanum tuberosum.

In some embodiments, the bacterial strain of the composition inhibits plant root infection by a pathogen. In some embodiments, the bacterial strain inhibits plant root disease by a pathogen. In some embodiments, the pathogen is Spongospora subterranea.

In some embodiments, the composition further comprises a biological agent. In some embodiments, the biological agent is a plant growth promoting agent. In some embodiments, the composition further comprises a bacterial stabilising agent. In some embodiments, the bacterial stabilising agent is xanthan gum.

In some embodiments, the composition is for use in promoting plant growth, promoting plant tuber yield, inhibiting plant root infection by a pathogen, and/or inhibiting plant root disease by a pathogen.

In a further aspect, the present invention provides a method for promoting plant growth, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In a further aspect, the present invention provides a method for promoting plant tuber yield, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In some embodiments, tuber size and/or weight is promoted.

In a further aspect, the present invention provides a method for inhibiting plant root infection by a pathogen, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In a further aspect, the present invention provides a method for inhibiting plant root disease by a pathogen, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In a further aspect, the present invention provides a method of promoting plant growth, of promoting plant tuber yield, of inhibiting plant root infection by a pathogen, or of inhibiting plant root disease by a pathogen, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In a further aspect, the present invention provides a composition for use in promoting plant growth, promoting tuber plant yield, inhibiting plant root infection by a pathogen, and/or inhibiting plant root disease by a pathogen, the composition comprising an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia.

In a further aspect, the present invention provides a composition when used for promoting plant growth, promoting plant tuber yield, inhibiting plant root infection by a pathogen, and/or inhibiting plant root disease by a pathogen, the composition comprising an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia.

In a further aspect, the present invention provides a method for promoting plant growth, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In a further aspect, the present invention provides a method for promoting plant tuber yield, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In a further aspect, the present invention provides a method for inhibiting plant root infection by a pathogen, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In a further aspect, the present invention provides a method for inhibiting plant root disease by a pathogen, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In a further aspect, the present invention provides a method of promoting plant growth, of promoting plant tuber yield, of inhibiting plant root infection by a pathogen, or of inhibiting plant root disease by a pathogen, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In a further aspect, the present invention provides a composition comprising:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10.

In a further aspect, the present invention provides a method for reducing the level of one or more pathogen stimulating compounds in the rhizosphere of a plant, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In a further aspect, the present invention provides a method of reducing the level of one or more pathogen stimulating compounds in the rhizosphere of a plant, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In a further aspect, the present invention provides a method for reducing the level of one or more pathogen stimulating compounds in the rhizosphere of a plant, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In a further aspect, the present invention provides a method of reducing the level of one or more pathogen stimulating compounds in the rhizosphere of a plant, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In some aspects, the one or more pathogen stimulating compounds are selected from L-glutamine and tyramine.

Other aspects and embodiments of the invention are described herein.

### BRIEF DESCRIPTION OF THE FIGURES

For a further understanding of the aspects and advantages of the present invention, reference should be made to the following detailed description, taken in conjunction with the accompanying figures which illustrate certain embodiments of the present invention.

FIG. 1—A flowchart depicting the steps involved in isolating and screening rhizosphere bacterial microorganisms that degrade L-Glutamine and L-Tyramine, in accordance with an embodiment of the present invention.

FIG. 2—An illustration of the experimental steps used in the identification and quantification of bacterial root colonization, including (I) bacterial count on the root surface, and (II) bacterial numbers inside root tissue.

FIG. 3—Photomicrographs of representative pure culture bacteria isolates from the rhizosphere of potato that showed growth ability in minimal media with Gln and Tyr as sole carbon sources. (Leica MZ12 stereoscope; magnification ×2; scale bar=10 mm; MSM agar).

FIG. 4—Graphs representing time-course monitoring of the growth of bacterial isolates (A) or metabolite degradation in M9 minimal medium enriched with 2 mM Gln or Tyr at 20 °C, 180 rpm for 96 hrs (B). Each value is the mean of results from 3 replicates±the standard error.

FIG. 5—Graphical representation of Tyr and Gln degradation by rhizosphere bacterial isolates of RR15 and RR09 strains measured using UPLC-MS/MS from 48 h culture in minimal media broth supplemented with 2 mM of each sole carbon source. Each data point represents the mean of three independent replicates. Different letters denote a significant treatment (α=0.05) effect for each compound.

FIG. 6—Ancestral taxonomic affiliation of Gln and Tyr degrading rhizosphere bacterial isolates inferred by using the Maximum Likelihood method and Tamura-Nei model. The percentage of replicate trees in which the associated taxa clustered together in the bootstrap test (1000 replicates) are shown below the branches. The branches in red indicate the rapid degraders used for further study while branches in blue are partial degraders used for comparison. Evolutionary analyses were conducted in MEGA X.

FIG. 7—Inferred ancestral states of candidate strain RR15 (A) and RR09 (B) using the Neighbor-Joining method. The tree shows a set of closest nucleotide sequences from the 16S rRNA database. All positions containing gaps and missing data were eliminated with a total of 223 and 125 positions, respectively, in the final data set. Evolutionary analyses were conducted in MEGA X.

FIG. 8—Graphs and images showing colonization of candidate isolates RR15 (E) and RR09 (D) in tissue-cultured roots. Progression of bacterial growth on the root surface assessed by (A) colony forming units (CFU), (B) internal roots assessed by qPCR 15, (C) in-vitro growth system and (D, E, F) Microscopic observation of colonization activity visualized through the root staining method of 6% aqueous aniline blue/glacial acetic acid (15:1:4, by volume).

FIG. 9—Comparisons of significantly reduced L-Glutamine and Tyramine levels in the soil exudates given the p-value threshold of 0.05, (A) Calculated average level in the rhizosphere between uninoculated and bacterial treated plants, (B) heatmap of available Gln and Tyr concentration following 8 weeks monitoring.

FIG. 10—Important metabolite features identified by ANOVA plot and Tukey's HSD with p value threshold of 0.05. The metabolites represented by red dots are significant, whereas the metabolites represented by green dots are not significant in differentiating the variables under investigation.

FIG. 11—Illustrations of the standing pot-soil growing system adopted in Example 2, consisting of experimental set-up for inoculum preparation, transplanting, and exudate collection and analysis, as well as recovery of amended microbial inoculants.

FIG. 12—Graphs showing the expression of metabolites selected by fold-change analysis with a given count threshold of 2.0. The data plotted represents a significant absolute value of change between the plant bacterial treatments and the control plants. The results are presented in log 2 scale, which indicates that the same fold change (up/down regulated metabolites) has the same distance from the zero baseline. Up/down regulated metabolites from zero baseline are shown by coloured dots, whereas grey dots represent no significant fold change.

FIG. 13—Heatmap analysis of the abundance of representative compounds found in the root exudates of control and bacterial-treated plants. (A) organic compounds, (B) amino acids, (C) glutamine and tyramine, alterations at 8 weeks post inoculation. Metabolite feature areas were normalized and range-scaled across all experimental samples.

FIG. 14—Graphs representing copy numbers of targeted bacterial 16S rRNA genes in rhizosphere and bulk soil at different sampling times. Absolute gene copy numbers were determined from a 5 ng of template DNA dilution curve series with known standards. Error bars are one standard error of the mean of replicate qPCR reactions.

FIG. 15—Total read abundance of the bacterial population present in the rhizosphere of Solanum tuberosum var. ‘ranger russet’ 8 weeks post inoculation, (A) Bar plot diagram showing OTU abundance (97% similarity) and assigned taxonomic classifications displayed at the phylum level for all individual samples; (B) Venn diagram illustrating the number of bacterial ASVs exclusive and common among control, bulk, and rhizosphere samples. ASVs in the Venn diagram were only included if they accounted for at least 0.1% of the total ASV in each sample.

FIG. 16—Differences in the relative abundance of bacterial taxa between the rhizosphere (with plants) and the bulk (no-plants) that were amended with bacterial isolates under study. The inner to outer rings represent the rhizosphere and bulk community, respectively. Different colours denote bacterial genera, and the percentage indicate the relative abundance of each sample type.

FIG. 17—Graphs showing the effect of single and combined bacterial inoculation on the growth of potatoes 8 weeks after planting. Bars at the top of each column denote standard deviation (n=5). Significant differences (p≤0.05) according to Tukey's HSD test are indicated by lower case letters across treatments.

FIG. 18—Schematic showing the exudate sampling technique from the plant-soil system employed in the study in Example 3.

FIG. 19—Graph showing total counts of strain Pantoea sp. RR15 in the rhizosphere of susceptible and resistant potato cultivars subjected to various treatments across 3 sampling periods. Bacterial copies represent the number of bacterial DNA per microliter as determined by qPCR. Values are expressed as means±SD from 3 biological and technical replicates.

FIG. 20—Graph showing glutamine and tyramine concentrations in soil as influenced by Pantoea RR15 treatment. The data reveal an average of glutamine and tyramine levels in cultivated soil at 4-, 12- and 21-weeks post-treatment. Bars within the same letter group are not statistically different (p<0.05, Tukey's HSD test).

FIG. 21—Heatmap showing metabolic transitions of targeted amino acids and organic compounds, the abundance of which is considerably increased or decreased in response to RR15 inoculation with or without S. subterranea. Data were compiled from 3 experimental replicates, with each sample being extracted and analysed in triplicate. The colour green denotes values that were all below the detection limit.

FIG. 22—Plots showing significant biochemical changes in targeted potato exudates from resistant and susceptible cultivars determined using fold change (FC) analysis. Each point on the figure was calculated using the p-value and fold-change values, which were set at an FC threshold of 2.0 and a t-test threshold of 0.5. The points that meet the criteria are highlighted in red and blue, respectively, indicating that they are strongly up/down regulated, whilst grey indicates that there is no significant fold change.

FIG. 23—Graphs showing the effects of plant growth promoting isolate Pantoea sp. RR15 on growth and development of resistant and susceptible potato cultivars 21 weeks post-treatment (Control=bacteria-free-pathogen-free; RR15=pathogen-free with bacteria; Sss+RR15=pathogen infected with bacteria; Sss=pathogen alone). Error bars show the standard deviation of the mean values of four replicates. Different letters on bars indicate statistically significant differences across treatment at p>0.05.

FIG. 24—Photographs showing tuber yield from resistant and susceptible varieties 21 weeks after treatment.

FIG. 25—Photographs depicting the effects of strain Pantoea RR15 on the vegetative growth of resistant and susceptible potato cultivars 4-, 12-, and 21-weeks post-inoculation.

FIG. 26—Photographs showing the extent of disease symptom development in roots and tubers of resistant and susceptible potatoes 21 weeks post bacterial treatment.

### DETAILED DESCRIPTION OF THE INVENTION

Nucleotide sequences are referred to herein by a sequence identifier number (SEQ ID NO:). A summary of the sequence identifiers is provided in Table 1. A sequence listing has also been provided at the time of filing this application.

TABLE 1
Summary of Sequence Identifiers

| Sequence | |
|---|---|
| Identifier | Description |
| SEQ ID NO: 1 | Nucleotide sequence of oligonucleotide primer 515F |
| SEQ ID NO: 2 | Nucleotide sequence of oligonucleotide primer 806R1 |
| SEQ ID NO: 3 | Nucleotide sequence of oligonucleotide primer |
| | RR15-2F |
| SEQ ID NO: 4 | Nucleotide sequence of oligonucleotide primer |
| | RR15-2R |
| SEQ ID NO: 5 | Nucleotide sequence of oligonucleotide probe |
| | RR15-2prb |
| SEQ ID NO: 6 | Nucleotide sequence of oligonucleotide primer |
| | RR09-1F |
| SEQ ID NO: 7 | Nucleotide sequence of oligonucleotide primer |
| | RR09-1R |
| SEQ ID NO: 8 | Nucleotide sequence of oligonucleotide probe |
| | RR09-1prb |
| SEQ ID NO: 9 | Nucleotide sequence of oligonucleotide primer 341F |
| SEQ ID NO: 10 | Nucleotide sequence of oligonucleotide primer 806R2 |

As set out above, the present invention is predicated, in part, on the isolation and characterization of rhizosphere bacterial isolates that have a significant affinity for the plant root rhizosphere. These pathogens have the capacity to degrade pathogen stimulating compounds within plant root exudates.

Accordingly, certain disclosed embodiments provide bacterial strains, compositions and methods that have one or more advantages. For example, some of the advantages of some embodiments disclosed herein include one or more of the following: novel isolated bacterial strains, including of the genus Pantoea, Rhodococcus, Bacillus, Brevibacterium, and Streptomyces; an isolated bacterial strain RR15 of the genus Pantoea deposited under Accession Number V22/011015 on 9 Jun. 2022 with the National Measurement Institute, Australia; cultures, supernatants and compositions containing the aforementioned strains; methods for promoting plant growth (including potato growth), and promoting plant tuber yield, using said strains; methods for inhibiting plant root infection by a pathogen, including potato root infection, using said strains; methods for inhibiting plant root disease by a pathogen, including potato root disease, using said strains; or the provision of a commercial alternative to existing compositions and methods. Other advantages of some embodiments of the present disclosure are provided herein.

Described herein are 12 bacterial isolates (designated as RR01, RR04, RR05, RR07, RR09, RR10, RR12, RR13, RR14, RR15, RR16, and RR17) that have been identified to populate the rhizosphere of plants and which show growth ability in minimal media supplemented with L-glutamine or tyramine as a sole carbon source. RR01, RR04, RR05, RR12, RR13, RR14, RR15, RR16, and RR17 are gram-positive bacteria, and RR07, RR09, and RR10 are gram-negative bacteria. Except for RR12, which formed a layer of hyphae appearing into a chain of spores, none of the other isolates were sporulating. The cells of the isolates varied in morphology from 0.3-0.9 μm in length and 0.5-1.2 μm in diameter. The gross morphology of these isolates was often irregular to circular in shape, with raised or convex margins, smooth to glistening colony surfaces, and a spectrum of white and yellow pigments. Cellular morphology included coccus to rod shaped cells, cell chains, individual cells, and a clustered organisation of cells, as summarised in Table 2 with respect to RR08 (of the genus Bacillus), RR09 (of the genus Rhodococcus), RR12 (of the genus Streptomyces), RR15 (of the genus Pantoea) and RR17 (of the genus Brevibacterium).

Bacterial strain Pantoea isolate RR15 demonstrates a strong capacity to degrade pathogen stimulating compounds (including L-glutamine or tyramine) within potato root exudates. Furthermore, this strain promotes plant development and growth in both resistant and susceptible cultivars validated by visible root improvement and increased plant biomass. In addition, plant tubers treated with RR15 bacterial inoculant showed enhanced emergence and early growth, and led to greater tuber yields, including increased tuber size, and increased tuber weight, compared to untreated (control) plants.

Accordingly, in a first aspect the present invention provides an isolated bacterial strain RR15 of the genus Pantoea, deposited under Accession Number V22/011015 on 9 Jun. 2022 with the National Measurement Institute, 1/153 Bertie Street, Port Melbourne 3207, Victoria, Australia. This deposit was made under the provisions of the Budapest Treaty on the International Recognition of the Deposit of Microorganisms for the Purpose of Patent Procedure and the Regulations thereunder. This assures maintenance of viable cultures for 30 years from the date of deposit. The organism will be made available by the National Measurement Institute under the terms of the Budapest Treaty which assures permanent and unrestricted availability of the organisms to the public upon issuance of the pertinent patent.

In the present invention, “isolated” refers to material removed from its original environment (e.g. the natural environment if it is naturally occurring), and therefore is altered “by the hand of man” from its natural state. For example, an isolated bacterial strain encompassed herein is one which has been removed from its natural growth environment (i.e. the plant rhizosphere roots and associated soil), by steps known in the art which include washing of plant roots, centrifugation of washed plant roots, resuspension of washed root pellet in appropriate media (such as a minimal salts media), and subsequent plating for single colony formation.

The isolated bacterial strain RR15 of the genus Pantoea may be referred to herein as “RR15”, “RR-15” and the like. RR15 is a gram-negative, yellow-pigmented bacterium with morphological characteristics set out in Table 2, when plated on 2.5% (w/v) agar in minimal salt medium supplemented with one or both of glutamine and tyramine. The bacterium will also grow on most common bacteriological media including Luria Broth agar, Tryptic soy agar, and Nutrient agar. The morphological profile of RR15 colonies includes a round form and convex elevation, smooth colony surface, opaque appearance, and yellow pigmentation with short rod-shaped cells occurring singly.

Reference herein to the aforementioned isolated bacterial strains includes mutants or variants of the strains, including mutants or variants of RR15. Such mutants and variants are therefore also encompassed by the present invention. Mutants can include, but are not limited to, spontaneous (“naturally-occurring”) mutant strains, or strains produced by mutagenesis of the parent strain by chemical means (such as treatment with N-methyl-N′-nitro-N-nitrosoguanidine or ethylmethanesulfone), through irradiation using gamma, x-ray, or UV-irradiation, or by other means known to a person skilled in the art. Variants of the bacterial strains described herein will have all the identifying characteristics of the bacterial strains described herein, and can be identified as having a genome that hybridizes under high stringency conditions to the genome of the parent bacterial strain. Variant strains can also include cell fusion strains and genetic recombination strains. Mutants and variants of the bacterial strains described herein retain the properties of the parent strain, and in some instances may have enhanced activity compared to the parent strain.

The present invention also includes a culture of one or more of the bacterial strains described herein, including a culture of bacterial strain RR15. A culture of the bacterial strains can be produced using standard techniques known in the art (see for example Ball A S, 1997, Bacterial Cell Culture: Essential Data, John Wiley & Sons, Inc.). Such cultures also form the basis of supernatant isolation as described below. A culture of one or more of the bacterial strains described herein may also comprise one or more additional bacterial strains such as those known to have a significant affinity for the plant root rhizosphere, have the capacity to degrade pathogen stimulating compounds within plant root exudates, and/or promote growth of the plant.

The bacterial strains described herein can be grown in nutrient media (also known as basal or complete media) that contains a carbon source for bacterial growth, water, various salts needed for bacterial growth and a source of amino acids and nitrogen (e.g., beef, yeast extract). This is an undefined medium because the amino acid source contains a variety of compounds with the exact composition unknown. Nutrient media contain all the elements that most bacteria need for growth and are non-selective, so they are used for the general cultivation and maintenance of bacteria kept in laboratory culture collections.

Bacterial cells can also be grown in minimal media. Minimal media include those media that contain the minimum nutrients possible for colony growth, generally without the presence of amino acids. For example, minimal medium may comprise: a carbon source for bacterial growth; various salts, which may vary among bacterial species and growing conditions, and which provide elements such as magnesium, nitrogen, phosphorous, and sulphur that assist the bacteria to synthesise protein and nucleic acid; and water.

In general, any rich media like NB (nutrient broth), LB (Luria-Bertani broth, Luria broth, lysogeny broth), TB (terrific broth), or minimal media with or without carbon and nitrogen added, can be used. The cells can be cultured at least at a temperature ranging from 20 °C (for slow growth) to 37 °C (for fast growth) in the presence of oxygen in appropriate flasks shaking at, for example, 150-300 rpm (preferably up to an OD₆₀₀ of 1.0). With respect to RR15, a sample of the strain obtained from a frozen source can be grown from a bacterial streak on a suitable bacteriological medium as described above (such as Luria Broth agar, Tryptic soy agar, and Nutrient agar). A single colony isolated from the bacterial streak can be inoculated in 100 mL of LB broth (10 g peptone, 5 g yeast, 5 g NaCl in 1 L of medium, pH 5.8) in 250 mL flasks in an orbital shaker set at 150 rpm and 23 °C until an OD₆₀₀ preferably up to 1 is reached. These parameters can be scaled up or down depending on the amount of culture of the bacterial strain that is required.

It has been shown herein that the bacterial strains encompassed by the present invention metabolise L-glutamine and tyramine. Therefore, in some embodiments the bacterial strains can be cultured in a medium comprising glutamine and/or tyramine as the sole carbon source. In such culture conditions, the concentration of glutamine and/or tyramine in the culture medium may be about 20 mM. Other concentrations are contemplated, as would be appreciated by a person skilled in the art.

The present invention also includes a supernatant obtained from a culture of one or more of the bacterial strains described herein, including a supernatant obtained from a culture of bacterial strain RR15. A supernatant can be obtained using standard techniques, such as using centrifugation of the culture of the bacterial cells to separate the cellular material following culturing. Typically, but not essentially, the supernatant can also be filtered after separation from the cellular component of the culture.

The cellular component once separated from the culture supernatant can also be stored as a cell extract for future use. Alternatively, a sample of the cultured cells in the form of a culture suspension may be stored for future use. In this regard, the cell culture sample may be stored on agar slopes for short periods of time or mixed with 50% glycerol and frozen at −80 °C for longer storage.

In some embodiments, the present invention provides a composition comprising a bacterial strain, culture of the strain, and/or a supernatant of the culture, as described above. As would be understood by a person skilled in the art, the amount of bacteria in the composition will be dictated by the nature of the composition, and the formulation of the composition with respect to its mode of intended application to a plant, as described further below. A composition, when in a solid form, such as in the form of a granule, particle or powder, can comprise stabilised bacterial cells mixed with an inert carrier including talc, vermiculite, perlite, diatomaceous earth, modified clays, etc. A composition, when in a liquid form, such as in the form of a cell suspension, solution, emulsion or aerosol, can comprise stabilised bacterial cells mixed with a suitable liquid carrier, as would be known in the art, and as described further below.

Compositions encompassed by the present invention can include excipients, also referred to herein as “acceptable carriers”. An excipient can be any material that an environmental sample (including soil and water samples), plant, and/or plant material can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Substances that enhance isotonicity and chemical stability of the composition are also examples of excipients. These can include various buffers (such as phosphate, bicarbonate, and Tris, buffers), and preservatives (such as thimerosal or o-cresol, formalin and benzyl alcohol). Excipients can also be used to increase the half-life of the composition, and include for example polymeric controlled release vehicles, biodegradable implants, liposomes, oils, esters, and glycols.

In some embodiments, a composition as described herein includes a bacterial stabilizing agent. The stabilizing agent is biocompatible and ensures that the bacterial strain remains active in the composition prior, and following, application. The stabilizing agent therefore does not diminish the activity of the bacterial strain. Suitable stabilizing agents would be known in the art, and include for example xanthan gum, cellulose derivatives, acrylic acid derivatives, modified clays and highly dispersed silica.

In some embodiments, a composition encompassed by the present invention further comprises a biological agent which enhances or complements the activity of the bacterial strain present in the composition. For example, the biological agent may be a further plant growth promoting agent such as a compound which has auxinic activity. Compounds with auxinic activity may be natural or synthetic, such as IAA (indole-3-acetic acid), 2,4-D (2,4-Dichlorophenoxyacetic acid) or NAA (1-Naphthaleneacetic acid). The biological agent may also be a pesticide.

Compositions encompassed by the present invention can be formulated in a manner dictated by the intended mode of application of the composition and dictated by the nature of the composition. Examples may include solutions, emulsions, suspensions, powders, foams, pastes, granules, sachets, aerosols, microencapsulations in polymeric substances, and ULV cold- and hot-fogging formulations. The nature of the formulation is not limited by the present invention. Depending on the formulation, the composition may require the presence of one or more of liquid solvents, pressurized liquefied gases, solid carriers, emulsifiers and/or dispersants and foam formers.

Examples of liquid solvents include aromatics such as xylene, toluene or alkyl-naphthalenes, chlorinated aromatics or chlorinated aliphatic hydrocarbons such as chlorobenzenes, chloroethylenes or methylene chloride, aliphatic hydrocarbons, such as cyclohexane or paraffins, for example mineral oil fractions, alcohols such as butanol or glycol, and their ethers and esters, ketones such as acetone, methyl ethyl ketone, methyl isobutyl ketone or cyclohexanone, strongly polar solvents such as dimethylformamide and dimethyl sulphoxide, and water, and also mineral, animal and vegetable oils such as, for example, palm oil or other plant seed oils.

Liquefied gaseous extenders or carriers are those liquids which are gaseous at normal temperature and under normal pressure, for example aerosol propellants such as halohydrocarbons and butane, propane, nitrogen and carbon dioxide.

Suitable solid carriers can include for example ground natural minerals such as kaolins, clays, talc, chalk, quartz, attapulgite, montmorillonite or diatomaceous earth, and ground synthetic minerals such as highly disperse silica, alumina and silicates. Suitable solid carriers for granules include for example crushed and fractionated natural rocks such as calcite, pumice, marble, sepiolite, dolomite, and synthetic granules of inorganic and organic metals, and granules of organic material such as sawdust, coconut shells, maize cobs and tobacco stalks.

Suitable dispersants and/or emulsifiers include nonionic, anionic or cationic dispersants or mixtures of nonionic or anionic dispersants. Suitable nonionic dispersants include ethylene oxide/propylene oxide block polymers, alkylphenol polyglycol ethers and tristyrylphenol polyglycol ethers and their phosphated or sulphated derivatives. Suitable anionic dispersants include lignosulphonates, salts of polyacrylic acid, and arylsulphonate/formaldehyde condensates.

Adhesives such as carboxymethylcellulose, natural and synthetic polymers in the form of powders, granules or latices, such as gum arabic, polyvinyl alcohol, polyvinyl acetate, and natural phospholipids such as cephalins and lecithins, and synthetic phospholipids, may also be used in the preparation of formulations of the compositions of the present invention. Colorants can also be included, such as inorganic pigments, for example iron oxide, titanium oxide, Prussian Blue, and organic dyestuffs, such as alizarin, azo and metal phthalocyanine dyestuffs, and trace nutrients, such as salts of iron, manganese, boron, copper, cobalt, molybdenum and zinc. Wetters may also be included in the composition when formulating. Suitable wetters include diisopropyl- or diisobutylnaphthalenesulphonates.

Formulations may also be in a controlled release format that is capable of slowly releasing the composition into the environment (including soil) or into or onto a plant or plant material or plant part. As used herein, a controlled release formulation comprises a composition of the present invention in a controlled release vehicle. Suitable controlled release vehicles include, but are not limited to, biocompatible polymers, polymeric matrices, capsules, microcapsules, microparticles, bolus preparations, osmotic pumps, diffusion devices, liposomes, and lipospheres. Preferred controlled release formulations are biodegradable (i.e. bioerodible).

A preferred controlled release formulation of the present invention is capable of releasing the composition to the target site preferably over a period of time ranging from about 1 to about 12 months. A preferred controlled release formulation is capable of effecting a treatment preferably for at least about 1 month, more preferably for at least about 3 months, even more preferably for at least about 6 months, even more preferably for at least about 9 months, and even more preferably for at least about 12 months.

The bacterial strains encompassed by the present invention have the ability to promote plant growth when applied to the plant. In this regard, the growth of the plant is increased relative to a control plant (for example a plant of the same origin which has not had the bacterial strain applied to it). A plant having an increase in growth is taken to mean a 1%, 2%, 3%, 4%, 5%, 10%, 20%, 30%, 40%, 50%, 60%, 70%, 80%, 90%, 100%, 2-fold, 3-fold, 4-fold, 5-fold, 10-fold, 20 fold, 30-fold, 40-fold, 50-fold, 6-fold, 70-fold, 80-fold, 90-fold, 100-fold, or greater, increase in growth compared to a control plant.

The term “when applied to the plant” in the context of the present invention is taken to mean an application of the bacterial strain to the plant in toto, an application of the bacterial strain to specific parts of the plant such as the roots, seeds or vegetative propagules (such as tubers), an application of the bacterial strain to the rhizosphere of the plant, and/or an application of the bacterial strain to soil in which the plant will be, or is, propagated in. Application of the bacterial strain encompasses application of the bacterial strain per se, application of a culture of the bacterial strain, application of a supernatant obtained from a culture of the bacterial strain, and/or application of a composition comprising one or more of these, as described above.

In some embodiments, plant root growth is an indicator of plant growth. Plant root growth can be measured with respect to root development, root length, root (fresh and dry) weight, and root architecture. In some embodiments, plant shoot growth is an indicator of plant growth. Plant shoot growth can be measured with respect to shoot development, shoot length, and shoot (fresh and dry) weight.

In some embodiments, harvestable products including tubers, corms, bulbs, flowers, seeds and fruits are an indicator of plant growth. Harvestable products can be measured with respect to weight, number, size and quality.

A plant having an increase in growth can therefore be a plant having any one or more of the following characteristics or phenotypes, each relative to a corresponding control plant: (i) increased root development; (ii) increased root length; (iii) increased root (fresh and dry) weight; (iv) increased shoot development; (v) increased shoot length; (vi) increased shoot (fresh and dry) weight; (vii) increased yield, weight, number, size and/or quality of tubers, corms bulbs or other vegetative products, and (viii) increased weight, number, size and/or quality of flowers, fruits, or seeds. Other parameters reflective of plant growth are also contemplated by the present invention. In this regard, examples of a plant having an increase in growth can include: (ix) an increased total dry matter weight; (x) an increased fresh mass; (xi) an increased root: shoot ratio; and (xii) an increased chlorophyll content per leaf.

Where the bacterial strains encompassed by the present invention have the ability to promote plant tuber yield when applied to the plant, tuber yield of the plant is increased relative to a control plant (for example a plant of the same origin which has not had the bacterial strain applied to it). In some embodiments, this may be seen as increased tuber size and/or increased tuber weight compared to untreated (control) plants. Accordingly, a plant having an increase in tuber yield is taken to mean a 1%, 2%, 3%, 4%, 5%, 10%, 20%, 30%, 40%, 50%, 60%, 70%, 80%, 90%, 100%, 2-fold, 3-fold, 4-fold, 5-fold, 10-fold, 20 fold, 30-fold, 40-fold, 50-fold, 6-fold, 70-fold, 80-fold, 90-fold, 100-fold, or greater, increase in tuber yield compared to a control plant.

A plant as referred to herein includes reference to all plant parts and plant populations, such as desired and undesired wild plants or crop plants (including naturally occurring crop plants). Crop plants may be plants which can be obtained by traditional breeding and optimization methods or else by biotechnological and recombinant methods, or combinations of these methods, including transgenic plants.

Plant parts are intended to mean all aerial and subterranean parts and organs of the plants, such as herb, pseudostem, shoot, leaf, bract, leaf sheaths, petiole, lamina, flower and root. Specific examples include leaves, needles, stalks, stems, flowers, fruiting bodies, fruit, roots, tubers, rhizomes, offshoots, suckers, and secondary growth. The plant parts also include crop material and vegetative and generative propagation material, for example cuttings, tubers, slips and seeds.

Examples of plants encompassed by the present invention include plant species and plant varieties, and their parts, which are found in the wild or which are obtained by conventional biological breeding methods, such as hybridization, meristem cultures, micropropagation, somatic embryogenesis, direct organogenesis or protoplast fusion. Reference to a plant may also include reference to transgenic plants and plant varieties which have been obtained by recombinant methods, if appropriate in combination with traditional methods (genetically modified organisms), for example, transformation by means of Agrobacterium or particle bombardment of embryogenic cells, and micropropagation. Preferred plants are those plant varieties which are commercially available or in use, such as plants producing fruits or vegetables or having other commercially valuable traits.

The plants which are encompassed by the present invention are those in which the bacterial strains of the present invention have the capacity to establish within the root rhizosphere of the plant. In some embodiments, the plants encompassed by the present invention are those which comprise L-glutamine and/or tyramine in exudates of roots of the plant. Root exudation is an important process determining plant interactions with the soil environment. Plant root exudates influence the constitution of the plant rhizosphere which is the narrow zone of soil surrounding the plant root. Methods for identifying the presence of glutamine and/or tyramine in exudates of roots of the plant are known in the art and include mass spectrometry-based applications.

Exemplary plants in accordance with the present invention include Solanaceae sp. (for example potatoes), cotton, flax, grapevine, vegetables and fruits (for example kiwi, pineapple), such as Rosaceae sp. (for example pome fruits such as apples and pears, but also stone fruits such as apricots, cherries, almonds and peaches, and soft fruits such as strawberries), or pomegranate from the genus of Punica, Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana plants and banana plantations as well as plantains), Rubiaceae sp. (for example coffee), Theaceae sp., Sterculiceae sp., Rutaceae sp. (for example citrus, lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers, melons, cucurbits, pumpkins), Alliaceae sp. (for example leeks, onions), Papilionaceae sp. (for example peas); major crop plants such as Gramineae sp. (for example corn, maize, turf, cereals such as wheat, rye, rice, barley, oats, sorghum, millet and triticale), Asteraceae sp. (for example sunflower), Brassicaceae sp. (for example cabbage such as white cabbage and red cabbage, broccoli, cauliflower, Brussels sprouts, pak choi, kohlrabi, small radishes, and also oilseed rape, mustard, horseradish and cress), Fabacae sp. (for example beans, peanuts), Papilionaceae sp. (for example soya beans), Chenopodiaceae sp. (for example sugar beet, fodder beet, Swiss chard, beetroot); useful plants and ornamental plants in gardens and forests; and in each case genetically modified types of these plants.

In some embodiments, the plant is of the Solanaceae family. The Solanaceae are a family of flowering plants that include annual and perennial herbs, vines, lianas, epiphytes, shrubs, trees, and a number of agricultural crops, medicinal plants, spices, weeds, and ornamentals. The Solanaceae family includes a number of commonly collected or cultivated species. The most economically important genus of the family is Solanum, which contains the potato (S. tuberosum), the tomato (S. lycopersicum), and the eggplant or aubergine (S. melongena). Another important genus, Capsicum, produces both chili peppers and bell peppers. In some embodiments, the plant is of the species Solanum tuberosum.

The bacterial strains encompassed by the present invention, when applied to the plant, have the ability to inhibit infection of plant roots (and/or other plant parts that exist below the ground) by a pathogen. The bacterial strains encompassed by the present invention, when applied to the plant, may also have the ability to inhibit disease of plant roots (and/or other plant parts that exist below the ground) by a pathogen. An inhibition can be measured as a decrease in infection of plant roots (and/or other plant parts that exist below the ground), or a decrease in disease in plant roots (and/or other plant parts that exist below the ground), relative to a control plant (for example a plant of the same origin which has not had the bacterial strain applied to it). A plant having a decrease in infection and/or disease of plant roots (and/or other plant parts that exist below the ground) is taken to mean a 1%, 2%, 3%, 4%, 5%, 10%, 20%, 30%, 40%, 50%, 60%, 70%, 80%, 90%, 100%, 2-fold, 3-fold, 4-fold, 5-fold, 10-fold, 20 fold, 30-fold, 40-fold, 50-fold, 6-fold, 70-fold, 80-fold, 90-fold, 100-fold, or greater, decrease in infection or disease compared to a control plant.

The extent of inhibition of infection and/or disease of plant roots (and/or other plant parts that exist below the ground) by a pathogen can be measured a number of ways and may depend on the plant type. Measures of infection and/or disease of plant roots (and/or other plant parts that exist below the ground) can include the presence and abundance of the invading pathogen within infected tissues, and/or the presence and extent of galls, rots, lesions, distorted or stunted growth, or other disease symptoms, on plant roots (and/or other plant parts that exist below the ground). In some embodiments where the plant is of the species Solanum tuberosum, the extent of plant root infection and plant root disease may be determined with reference to the presence of tuber lesions or measurement of tuber yield (size and number of potato tubers).

The presence and extent of a pathogen in the plant roots (and/or other plant parts that exist below the ground) may also be detected using molecular biology-based approaches. These include the use of antibodies specific for the pathogen under investigation and detection of antibody binding using immunological assays such as ELISA-based assays. PCR-based assays may also be used wherein oligonucleotide primers specific for a pathogen under investigation are used to amplify DNA obtained from plant roots for example.

In the context of the present invention, reference to a pathogen encompasses any pathogen that can infect plant roots (and/or other plant parts that exist below the ground). Plant pathogens are microorganisms that belong to the taxonomic groups which include bacteria, viruses, fungi, protozoa, and nematodes.

Most phytopathogenic fungi belong to the Ascomycetes and the Basidiomycetes. The fungi reproduce both sexually and asexually via the production of spores and other structures. Spores may be spread long distances by air or water, or they may be soil borne. Many soil inhabiting fungi are capable of living saprotrophically, carrying out the part of their life cycle in the soil. Biotrophic fungal pathogens colonize living plant tissue and obtain nutrients from living host cells. Necrotrophic fungal pathogens infect and kill host tissue and extract nutrients from the dead host cells. Significant fungal plant pathogens of the Ascomycetes include Fusarium spp., Thielaviopsis spp., Verticillium spp., Magnaporthe grisea, and Sclerotinia sclerotiorum. Significant fungal plant pathogens of the Basidiomycetes include Ustilago spp., Rhizoctonia spp., Phakospora pachyrhizi, Puccinia spp., and Armillaria spp. Fungus-like organisms are also pathogenic. These include oomycetes such as those of the genus Phytophthora spp., and Pythium spp., Chytridomycetes such as those of the genus Olpidium, and phytomyxea such as those of the genus Plasmodiophora and Spongospora, are also examples of soil-borne pathogens.

Most bacteria that are associated with plants are actually saprotrophic and do no harm to the plant itself. However, a small number of species are pathogenic and cause disease. Most plant pathogenic bacteria are rod-shaped (bacilli). In order to be able to colonize the plant they have specific pathogenicity factors. Five main types of bacterial pathogenicity factors are known and include cell wall degrading enzymes, toxins, effector proteins, phytohormones and exopolysaccharides. Pathogens such as Erwinia species use cell wall degrading enzymes to cause soft rot. Agrobacterium species change the level of auxins to cause tumours with phytohormones. Exopolysaccharides are produced by bacteria and block xylem vessels, often leading to the death of the plant. Significant bacterial plant pathogens include Agrobacterium spp., Burkholderia spp., Ralstonia spp., Pseudomonadota, Xanthomonas spp., Pseudomonas spp., Pectobacterium spp., Dickeya spp., Clavibacter spp., Streptomyces spp., Xylella spp, and Candidatus Liberibacter spp.

Plant viruses are obligate intracellular parasites that do not have the molecular machinery to replicate without a host. Plant viruses can be pathogenic to higher plants. Most plant viruses are rod-shaped, with protein discs forming a tube surrounding the viral genome; isometric particles are another common structure. They rarely have an envelope. The great majority have an RNA genome, which is usually small and single stranded (ss), but some viruses have double-stranded (ds) RNA, ssDNA or dsDNA genomes. To transmit from one plant to another and from one plant cell to another, plant viruses must use strategies that are usually different from animal viruses. Most plants do not move, and so plant-to-plant transmission usually involves vectors (such as insects). Examples of plant viruses include Tobacco mosaic virus, Tomato spotted wilt virus, Tomato yellow leaf curl virus, Cucumber mosaic virus, Potato virus Y, Cauliflower mosaic virus, African cassava mosaic virus, Plum pox virus, Brome mosaic virus, Potato virus X, Citrus tristeza virus, Tobacco rattle virus, Barley yellow dwarf virus, Potato leafroll virus and Tomato bushy stunt virus.

Nematodes are small, multicellular, wormlike animals. Many live freely in the soil, but there are some species that parasitize plant roots. The plant parasitic nematodes belong to several genera and can have differing parasitic lifestyles including both endoparastic (live within the plant) and ectoparasito (live and feed externally). Endoparasites may be migratory (readily move within the invaded tissues), or sedentary (remain in place once infection has occurred). Some nematodes also have the capacity to transit plant viruses. Globodera pallida and G. rostochiensis are examples of potato cyst nematodes which cause significant damage in Europe every year. Cyst nematodes are sedentary root feeding endoparasites. Root knot nematodes such as Meloidogyne spp. also common sedentary root feeding endoparasites that have quite a large host range. Root knot and cyst nematodes are able to cause radical changes in root cells in order to facilitate their lifestyle. Lesion nematodes such as Pratylenchus spp. are plant root ectoparasites and commonly affect a wide range of plant species.

In some embodiments, the pathogen is Spongospora subterranea f.sp subterranea (Sss) which causes powdery scab disease in potatoes. Spongospora subterranea infects the below ground structures of the host plant, Infection leads to hypertrophy and hyperplasia of the host cells and eventual bursting. Zoospores infect the root hairs by attaching to the outer surface, encysting, and then penetrating the epidermis. Once inside, the multinucleate plasmodium divides to form a zoosporangium which in turn will produce secondary zoospores that will be released to facilitate further infections. Root infection can occur at all stages of plant development. In later stages of root infection infected host cells enlarge into a gall. Within the root galls aggregates of resting spores form, and are termed sporosori. Eventually the gall swells and bursts out the epidermis of the tuber, releasing the spores back into the soil. Gall severity depends on inoculum level, soil environment, and potato cultivar. Tuber infection is most prevalent in the early stages of tuber formation while the potato tissue is unsuberized. Blister like lesions form within which sporosori form. As the lesions mature they rupture releasing sporosori inoculum into the soil. The sporosori within tuber lesions look like a powdery mass, which gives the associated disease its name.

As indicated above, the bacterial strains encompassed by the present invention have the ability to promote plant growth. Accordingly, the present invention provides a method for promoting plant growth, the method comprising applying to the plant or to soil in which the plant will be, or is, propagated in, an effective amount of the bacterial strain, culture of the bacterial strain, supernatant of the culture, and/or a composition comprising these, as described above.

The term “applying to the plant” as used herein has the same meaning as the term “when applied to the plant” as defined above. In effect, the method is carried out directly on the plant as a whole or on parts of the plant, or by action on the surroundings of the plant, such as the soil in which the plant is propagated, the root rhizosphere, or general habitat of the plant. Application to the plant of the bacterial strain, culture of the bacterial strain, supernatant of the culture, and/or a composition comprising these, may be achieved using standard treatment methods such as spraying, dipping, atomizing, irrigating, dusting, fogging, foaming, painting, spreading on, watering, drip irrigating, and the like. The material to be applied will be formulated accordingly, as described above.

As indicated above, the bacterial strains encompassed by the present invention have the ability to promote plant tuber yield. Accordingly, the present invention provides a method for promoting plant tuber yield, the method comprising applying to the plant or to soil in which the plant will be, or is, propagated in, an effective amount of the bacterial strain, culture of the bacterial strain, supernatant of the culture, and/or a composition comprising these, as described above.

As indicated above, the bacterial strains encompassed by the present invention have the ability to inhibit plant root infection by a pathogen, and inhibit plant root disease by a pathogen. Accordingly, the present invention provides a method for inhibiting plant root infection by a pathogen, or inhibiting plant root disease by a pathogen, the method comprising applying to the plant or to soil in which the plant will be, or is, propagated in, an effective amount of the bacterial strain, culture of the bacterial strain, supernatant of the culture, and/or a composition comprising these, as described above.

In some embodiments, inhibiting plant root infection and plant root disease can be effected by treating propagating material of the plant with the bacterial strain, culture of the bacterial strain, supernatant of the culture, and/or a composition comprising these. Plant propagating material refers to any part or product of a plant that alone or in combination with other parts or products of that plant produces another plant with the same essential characteristics. In this regard, propagating material may include seeds, cuttings, seedlings, and the like.

Treating the propagating material of the plant protects the material, and the germinating plant, from soil-borne pathogens such that the immediate treatment of the plant crop at the time of planting, or shortly thereafter, can be avoided. In the case of treating seed of the plant, the material to be applied will preferably be formulated as a dry powder for dry seed treatment, a solution for seed treatment, a water-soluble powder for slurry treatment, by seed incrusting, by seed coating, and the like.

In some embodiments of the methods described herein, the bacterial strain, culture of the bacterial strain, supernatant of the culture, and/or a composition comprising these, can be applied to soil in which the plant is growing, in which the plant will be propagated in, or in which propagating material of the plant will be grown in. Alternatively, soil can be pre-prepared to contain the relevant material, such as in a slow-release formulation. Application of the relevant material to the soil in which the plant is growing can be achieved by any suitable means as described above, for example by spraying plant crops, irrigating crops, watering crops, drip irrigating crops, and the like.

The methods for inhibiting plant root infection and inhibiting plant root disease as described herein can be carried out curatively as well as preventively.

As indicated above, the methods described herein require applying an effective amount of the bacterial strain, culture, supernatant or composition comprising one or more of these. An effective amount is the quantity of the bacterial strain, culture, supernatant or composition which, when administered to a plant, promotes the growth of the plant, inhibits plant root infection and/or inhibits plant root disease. The amount to be administered to a plant will depend on the particular characteristics of the bacterial strain, the plant (or soil) to be treated, and the nature of the formulation of the material. A person skilled in the art will be able to determine appropriate dosages depending on these and other factors. The effective amount of the bacterial strain, culture, supernatant or composition comprising one or more of these, of the present invention is not particularly limited.

For the treatment of plant parts, an effective amount of the relevant material to be applied will generally be between 0.1 to 10,000 g per hectare. For the treatment of propagating material of the plant, an effective amount of the relevant material to be applied will generally be between 0.001 to 50 g per kilogram of propagating material. For the application to soil in which the plant is growing, in which the plant will be propagated in, or in which propagating material of the plant will be grown in, an effective amount of the relevant material to be applied will generally be between 0.1 to 10,000 g per hectare.

The following aspects of the invention reflect particular embodiments of the invention and are not limiting on the scope of the invention as described herein.

In a second aspect the present invention provides a composition comprising an isolated bacterial strain RR15 of the genus Pantoea, deposited under Accession Number V22/011015 on 9 Jun. 2022 with the National Measurement Institute, Australia.

In a third aspect the present invention provides a composition comprising a culture of an isolated bacterial strain RR15 of the genus Pantoea, deposited under Accession Number V22/011015 on 9 Jun. 2022 with the National Measurement Institute, Australia.

In a fourth aspect the present invention provides a composition comprising a supernatant of a culture of an isolated bacterial strain RR15 of the genus Pantoea, deposited under Accession Number V22/011015 on 9 Jun. 2022 with the National Measurement Institute, Australia.

In a fifth aspect the present invention provides a method for promoting plant growth, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In a sixth aspect the present invention provides a method for promoting plant tuber yield, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In a seventh aspect the present invention provides a method for inhibiting plant root infection by a pathogen, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In an eighth aspect the present invention provides a method for inhibiting plant root disease by a pathogen, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In a ninth aspect the present invention provides a composition for use in promoting plant growth, promoting plant tuber yield, inhibiting plant root infection by a pathogen, and/or inhibiting plant root disease by a pathogen, the composition comprising an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia.

In a tenth aspect the present invention provides a composition when used for promoting plant growth, promoting plant tuber yield, inhibiting plant root infection by a pathogen, and/or inhibiting plant root disease by a pathogen, the composition comprising an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia.

In an eleventh aspect the present invention provides a method for promoting plant growth, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In a twelfth aspect the present invention provides a method for promoting plant tuber yield, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In a thirteenth aspect the present invention provides a method for inhibiting plant root infection by a pathogen, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In a fourteenth aspect the present invention provides a method for inhibiting plant root disease by a pathogen, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In a fifteenth aspect the present invention provides a composition comprising:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10.

As indicated above, the present invention relates to bacterial strains which have the ability to degrade pathogen stimulating compounds (such as L-glutamine and tyramine) within plant root exudates of the rhizosphere of a plant. Accordingly, in a sixteenth aspect the present invention provides a method for reducing the level of one or more pathogen stimulating compounds in the rhizosphere of a plant, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

In a seventeenth aspect the present invention provides a method for reducing the level of one or more pathogen stimulating compounds in the rhizosphere of a plant, the method comprising applying to the plant or to soil in which the plant will be, or is, propagated in, an effective amount of:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10; or
(iv) a composition comprising (i), (ii), and/or (iii).

In some embodiments of the sixteenth or seventeenth aspects of the invention, the one or more pathogen stimulating compounds are selected from L-glutamine and tyramine.

With respect to the second to seventeenth aspects of the invention, the nature of plant growth promotion, plant tuber yield promotion, plant root growth promotion, the plant family and species, plant root infection inhibition, plant root disease inhibition, the pathogen, biological agent, plant growth promoting agent, bacterial stabilizing agent, the mode of composition application to the plant, and the like, are described above with respect to the first aspect of the invention.

The invention is further illustrated in the following examples. The examples are for the purpose of describing particular embodiments only and are not intended to be limiting with respect to the above description.

### Example 1

### Selection of Rhizospheric Bacteria that Degrade Root Exudate Compounds Associated with Pathogen Germination and Chemotaxis

Root exudation is associated with mediating interactions between plants and diverse soil biotas including rhizosphere microbial populations, and arguably less commonly, with plant pathogens. Potato-root exudates, such as L-Glutamine (Gln) and Tyramine (Tyr), have previously been demonstrated to aid in the germination of the resting spores of the plasmodiophorid pathogen, Spongospora subterranea. The aim of this study was to isolate and characterise bacteria capable of degrading Gin and Tyr within the potato rhizosphere and hence indirectly inhibit pathogen infection processes. This is the first study to describe selection of rhizosphere bacteria capable of degrading potato stimulatory compounds as a target against germination and attraction of a plant pathogen.

### Materials and Methods

All media and buffers used were of analytical grade and were purchased from Sigma-Aldrich Pty Ltd. (St. Louis, MO, USA) unless otherwise stipulated. Assay preparations were diluted with Milli-Q (Merck kGaA, Darmstadt, Germany) deionized purified water and autoclaved.

### Isolation of Culture and Initial Screening

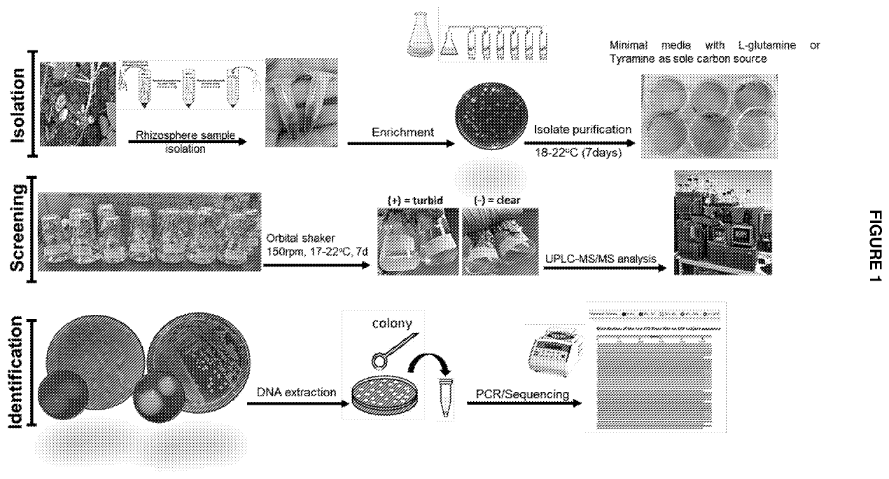

Rhizosphere samples were collected from five randomly selected plants within a commercial crop of Solanum tuberosum cv. ‘Ranger Russet’ growing in North West Tasmania (lat-41.17, long 146.33). Potato plants were sampled at ~75 days after planting. Plant roots were shaken to remove loosely adhering soil; the remaining soil firmly adhered to the roots was considered as the rhizosphere soil (Barillot C D C et al., 2012, Annals of Microbiology, 63(2): 471-476). The roots were rinsed four times in a conical flask containing saline distilled water (0.85% NaCl) and vortexed three times for 30 s. Following that, cleaned roots were transferred to 50 mL falcon tubes containing 30 mL phosphate-buffered saline solutions (PBS, 0.1 M) and centrifuged for 15 minutes at 25 °C at 12,000×g. The supernatant was discarded, and the resultant rhizosphere soil pellet was collected and stored at 4 °C until use (Barillot C D C et al., 2012, supra; Buermans H O and den Dunnen J T, 2014, Biochim Biophys Acta, 1842(10): 1932-1941). To isolate bacteria, 0.2 g of pelleted rhizosphere soil was added to 50 mL sterile minimal salt media (MSM) broth and then the mixture was centrifuged at 3000 g for 5 min. The supernatant was serially diluted with MSM from 10⁻¹ to 10⁻⁸ fold. An aliquot of 150 μl was spread onto agar containing either filter sterilized 1× stock of Gln (2.92 mg L⁻¹) or Tyr (2.74 mg L⁻¹) in 2.5% (w/v) agar in MSM. Plates were inverted and incubated at 18-23 °C with growth monitored. Bacteria with morphologically distinct colonies and pigmentation were chosen for sub-culturing, purification, and re-streaking onto fresh agar plates (FIG. 1). To confirm purity of the culture, a standard microbiological evaluation was conducted that involved streaking on nutrient agar, macroscopic and microscopic observation of colony and cell morphology via wet-mounting and Gram staining reaction in accordance with Bergey's Manual of Systematic Bacteriology of Archaea and Bacteria (De Vos P G et al., 2009, Bergey's Manual of Systematic Bacteriology: Volume 3. The Firmicutes). Pure cultures were maintained in 50% glycerol and kept at −80 °C.

### Identification of Isolates

### Colony and Cell Morphology

Visual observation of colony growth on a solid medium was assessed using a Leica MZ12 stereomicroscope (2× magnification). Gram staining was conducted on cells collected from fully developed colonies, and cellular morphology was evaluated at 400× magnification with a Leica DMLB compound microscope (Lieca Microsystem, Wetzlar GmbH, Germany).

### Molecular Typing and Phylogenetic Analysis

The dNeasy ultra-clean microbial kit (Qiagen, Germany) was used to extract genomic DNA from 48-hour exponentially growing cultures following the manufacturers protocols. DNA extracts were then used for 16S rRNA gene amplification with primers 515F: 5′-GTGCCAGCMGCCGCGGTAA-3′ (SEQ ID NO: 1) and 806R1: 5′-GGACTACHVGGGTWTCTAAT-3′ (SEQ ID NO: 2). The reactions were carried out on a PCR thermocycler (Eppendorf Master cycler) using a thermocycle profile of 95 °C (5 min) followed by 30 cycles of 95 °C (1 min), 65 °C (1 min), 72 °C (1.5 min); with a final extension of 72 °C (10 min). PCR products were visualized under blue light following electrophoresis in a 1% agarose gel and 0.01% SYBR Safe DNA gel stain (Invitrogen, USA) and purified using a QIAquick PCR Purification Kit (Qiagen, Germany) following the manufacturer's guidelines. The quality and concentration of extracted gDNA were determined using the Qubit® 2.0 Fluorometer (Life technologies, Darmstadt, Germany), and sent to the Australian Genome Research Facility for sequencing (http://www.agrf.org.au/). To determine bacterial putative identities, a homology search was performed using the GenBank DNA database (http://www.ncbi.nlm.nih.gov) by the BLAST program. Nucleotide substitution rates and bacterial sequence alignment was defined by ClustalW software (Thompson J D et al., 1994, Nucleic Acids Res., 22:4673-4680). Evolutionary analyses were tested using MEGA X bootstrapping with 1000 replications (Saitou N and Nei M, 1987, Mol. Biol. Evol., 4(4): 406-425). Phylogenetic trees were drawn to scale with branch lengths in the same units as those used to infer the phylogenetic tree. The evolutionary distance was computed using the Maximum likelihood and Neighbour-Joining approach conducted in MEGA X (Tamura K et al., 2004, PNAS, 101:11030-11035).

### Test of Bacterial Degradation Characteristics

To achieve uniform inoculum size, bacterial isolates that grew well with Gln or Tyr as the sole carbon source were enriched separately by inoculating them into 100 mL of LB broth (10 g peptone, 5 g yeast, 5 g NaCl in 1 L of medium, pH 5.8) in 250 mL flasks in an orbital shaker set at 150 rpm and 23 °C until OD₆₀₀ was equal to 1 (Lami M J et al., 2020, J. Appl. Microbiol., 129(5): 1321-1336). Thereafter, bacterial cells were harvested by centrifuging at 6000 g for 5 minutes at 4 °C. The procedure was then repeated three times under similar circumstances, with 1 mL of culture solution withdrawn and reconstituted in a fresh 100 mL MSM media, this was then utilised as the starting culture for further studies (Krishnan M et al., 2018, Sci. Rep., 8(1): 2609. doi10.1038/s41598-018-20718-1). The degradation potential of selected isolates was investigated by introducing a 100 μL of active inoculum from each bacterial isolate adjusted to a set value of OD₆₀₀=0.110⁻⁸ CFU mL⁻¹ in 50 mL MSM supplemented with 20 mM filter sterilised Tyr and Gln (Millex-GS Syringe Filter Unit, 0.22 μm). All flasks were incubated at 18-23 °C on an orbital shaker set to 150 rpm. The uninoculated sterilized medium was treated as a control reference to check the abiotic degradation under the same conditions (Mahboubifar M et al., 2010, Food Analytical Methods, 4(2): 150-154). Periodically, a 1 mL aliquot of inoculated broth was removed to determine turbidity via OD on a spectrophotometer (λ=600 nm) for bacterial growth monitoring, while residual compounds in culture samples were transferred to falcon tubes and centrifuged for 5 min at 12,000×g, with supernatant collected and transferred to a collection tube for reverse-phase ultra-pressure liquid chromatography (UPLC) analysis. For all experiments, each test was carried out in triplicate.

### Estimating Root Colonizing Ability In Vitro

Two bacterial strains (designated RR15 and RR09) were examined for their ability to colonise potato roots of three-week-old tissue cultured potato plants (cv. Ranger Russet). The roots of 36 potato plants (6-time points×2 bacterial isolates×3 replicates) were suspended in 20 mL liquid potato medium and treated with a suspension of 10⁻⁸ CFU mL⁻¹ of either bacterial isolate, which was then sampled at 24-hour intervals over 15 days (3 replicates for each sampling). External (root surface) and internal (within root tissues) populations of inoculated bacteria were quantified on potato multiplication liquid medium on days 1, 3, 6, 9, 12, and 15 post inoculation.

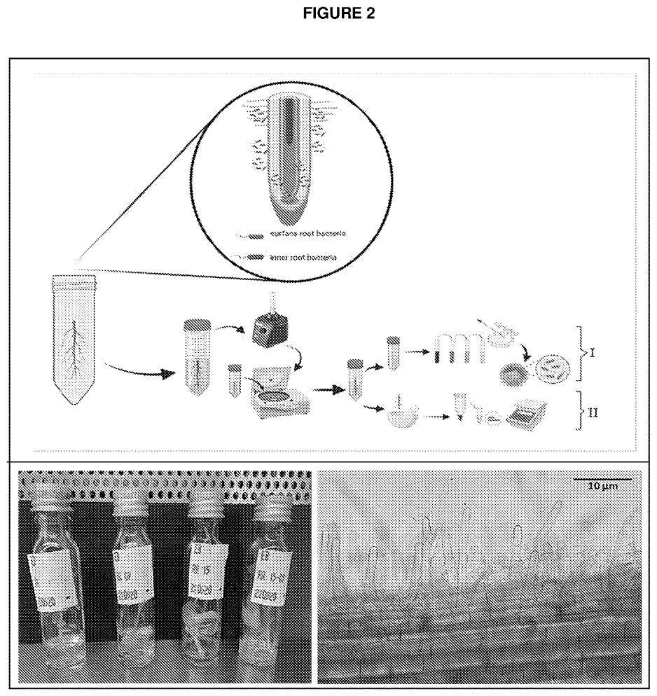

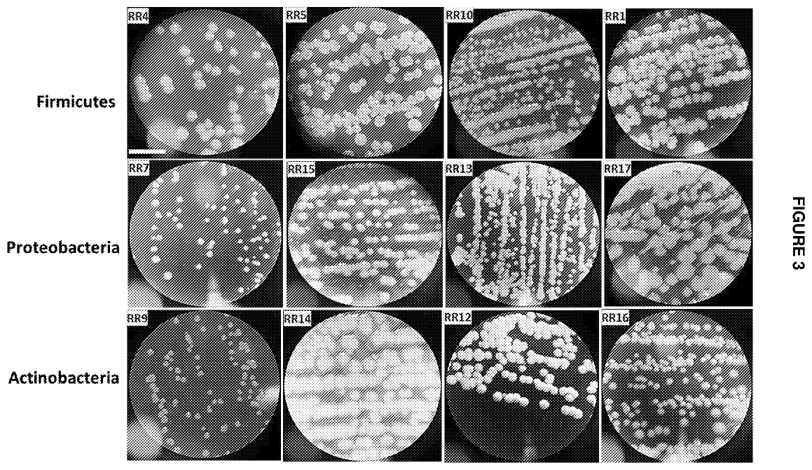

To determine root surface colonization, the roots of each replicate plant were cut, weighed, and placed into 50 mL Falcon tubes containing 25 mL of sterile phosphate-buffered saline solution (pH 7.4). After vortexing the tubes for 5 minutes at 5000×g, the suspensions were collected and successive dilutions of the suspension were plated on LB agar. The number of viable cells was then counted and expressed as CFU per gram root. Meanwhile, the same roots were utilised again to identify root colonisation. The roots were then surface sterilised in three steps: a 60 s ethanol wash, a 6 min 3.1% sodium hypochlorite (NaOCl) wash, a 30 s ethanol wash, and a final rinse in sterile Milli-Q water. To confirm bacterial colonisation inside root tissues, root segments were macerated, and homogenates were placed in a collecting tube. DNA was isolated from the homogenates after centrifugation at 3000 g for 10 mins at 4 °C (FIG. 2). Total DNA extraction was performed using the Qiagen dNeasy Power Plant Pro kit.

Amplification of 250-300 bp using primers unique to the target strains was accompanied by in planta quantification of two candidate isolated strains. QPCR primers and TaqMan probes were designed from the 16S region of each isolate using Primer Quest software. Primers RR15-2F (ACGGCCGCAAGGTTAAA) (SEQ ID NO: 3) and RR15-2R (GCTGGATGTCAAGAGTAGGTAAG) (SEQ ID NO: 4) and probe RR15-2prb (ACAAGCGGTGGAGCATGTGGTTTA) (SEQ ID NO: 5) were used to detect and enumerate isolate RR15, while primers RR09-1F (CGGAATCACTGGGCGTAAA) (SEQ ID NO: 6) and RR09-1R (GTTCCACCAACCTCTACCATAC) (SEQ ID NO: 7), and probe RR09-1prb (CGGAATCACTGGGCGTAAA) (SEQ ID NO: 8) were used to monitor isolate RR09. To normalise qPCR results for quantitative analysis, the marine bacterium Pseudoaltemoranas prydzensis was employed as a positive internal reference (Bowman J P, 1998, Int. J. Syst. Bacteriol., 48 Pt3, 1037-1041). The Rotor-Gene Q system (Qiagen, Hilden, Germany) was used to perform qPCR using 10 ng of bacterial DNA and the HEX probe. Three biological replicates each with three technical replicates were used to analyse each sample. The basic curve method, as defined for qPCR analysis, was used to analyse relative quantification.

### Assessment of Metabolite Degradation by Bacterial Isolates In Situ

A total of 64 potato plants were grown in a glasshouse maintained at 20±2 °C for an 8-week period. Four pots from each group (untreated control and bacteria-treated plants) were randomly selected for exudate collection at 2, 4, 6, and 8 weeks after bacterial inoculation. Root exudate metabolites from the potted plants were collected through a hole in the centre of the conically shaped bottom of the pot by firstly saturating the soil with 300 ml distilled water for 2 hours and then collecting an aliquot of 100 ml of leachate from the pot (hereafter referred to as the root exudate). Leachates were shaken at 100 rpm for 10 min, then centrifuged for 10 min at 5000×g. The supernatant containing water-soluble components was filtered sterilized through a 0.22 μm millipore filter, frozen, and stored at −20 °C before analysis by UPLC-tandem mass spectrometry (UPLC-MS/MS), which targeted 16 common amino acids (aAs) and 23 common organic compounds (oCs) previously found in potato plant soil-root exudates (Balendres M A et al., 2016a, J. Agric. Food Chem., 64(40): 7466-7474; Balendres M A et al., 2016b, Australasian Plant Pathology, 45(3): 229-240).

### Statistical Analysis

The mean differences between treatments were examined using ANOVA and Tukey's HSD post-hoc tests with a significance threshold of (p≤0.05) in R for SPSS Statistics version 28.0.0.0. (SPSS Inc., NY, USA). The values of the metabolites data were log 10 transformed to meet assumptions of normality and homogeneity of variance in ANOVA using R package MetaboAnalystR (https://www.metaboanalyst.ca/).

### Results

### Characterisation of Bacterial Isolates

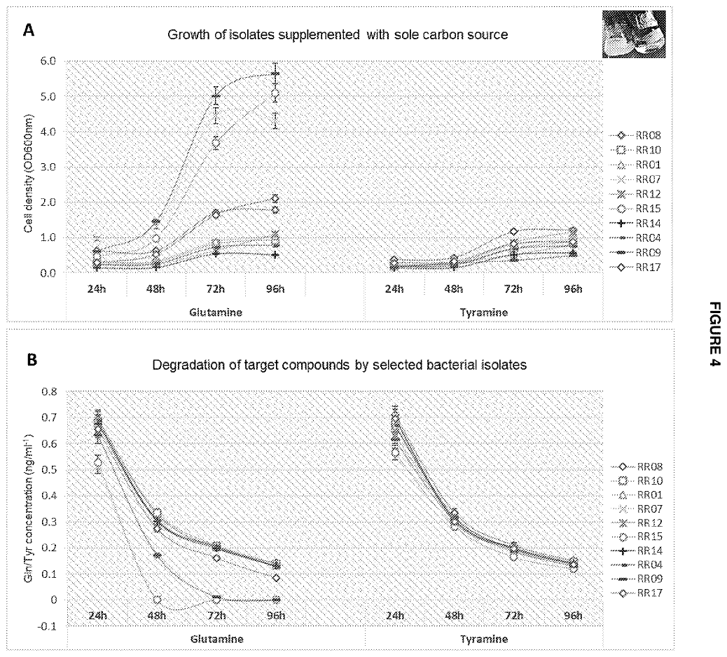

The gross morphology of representative bacterial strains found to have grown on minimal media amended with Gin and Tyr as sole carbon sources was often irregular to circular in shape, with raised or convex margins, smooth to glistening colony surfaces, and a spectrum of white and yellow pigments. Cellular morphologies included coccus to rod shaped cells, cell chains, individual cells, and clustered organisation of cells (FIG. 3; Table 2).

Bacterial isolates RR01, RR04, RR05, RR16, RR17, RR12, RR13, RR14, and RR15 were gram-positive bacteria, whereas isolates RR07, RR09, and RR10 were gram-negative bacteria. Except for RR12, which formed a layer of hyphae appearing into a chain of spores, none of the other isolates were sporulating. The cells of the isolates varied in morphology from 0.3-0.9 μm in length and 0.5-1.2 μm in diameter.

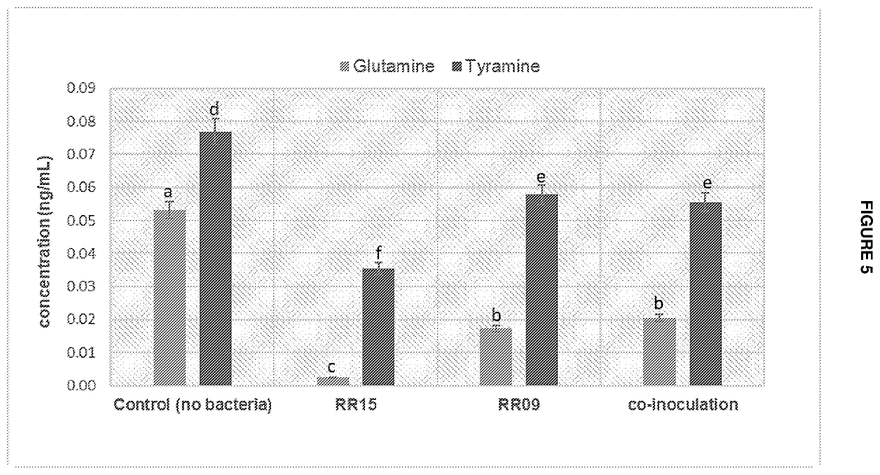

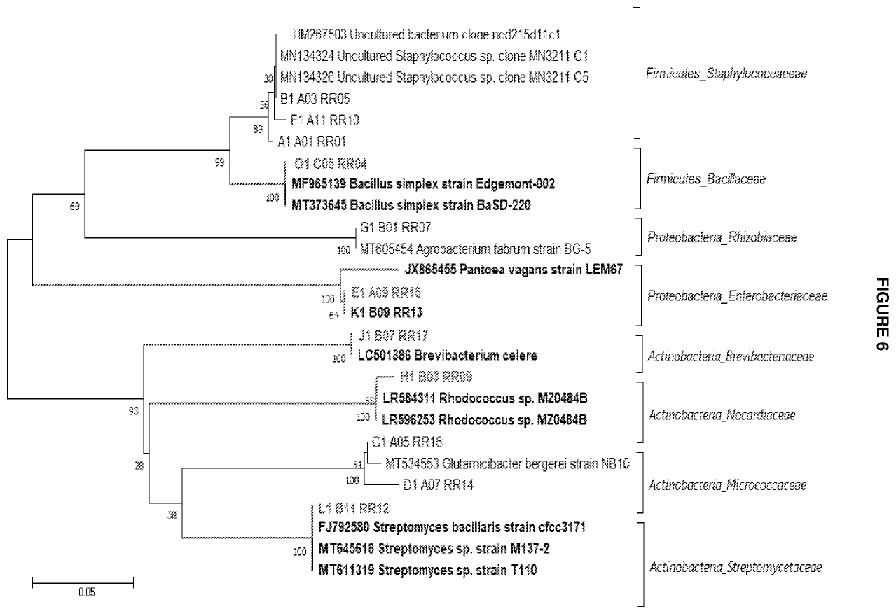

As shown in FIG. 4, bacteria cultured in minimal media supplemented with 20 mM Gln or Tyr exhibited significant variation in growth as measured by the turbidity of the culture medium. The bacterial assays with Gln as a supplement appeared thick and cloudy, but the assays with Tyr as a supplement were less cloudy even after 96 h. In the presence of Gln, isolates RR17 (OD₆₀₀=2.1), RR15 (OD₆₀₀=5.1), RR09 (OD₆₀₀=5.6) grew faster than the other isolates tested (FIG. 4A-left graph), which led to more rapid depletion of Gln from the medium (FIG. 4B—left graph). On the other hand, all isolates grew relatively poorly in the Tyr supplemented medium (FIG. 4A—right graph) as evidenced by the less than 100% breakdown of Tyr during the incubation period (FIG. 4B—right graph).

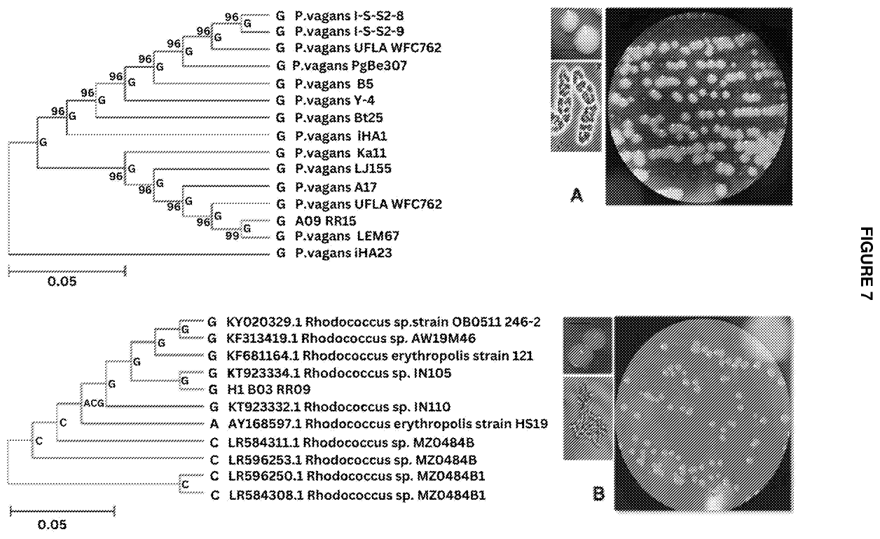

As shown in FIG. 5, glutamine remaining in the supernatant of the RR15 bacterial assay (0.002±0.003 ng per mL⁻¹ hour⁻¹), is undetectable within the culture medium after 48 hours, whereas isolate RR09 (0.017±0.009 ng per mL⁻¹ hour⁻¹) took 96 hours to reach similar glutamine substrate degradation levels. Tyramine degradation by both isolates was slower than for glutamine, however isolate RR15 degraded tyramine faster than isolate RR09 with an average of 0.035±0.019 ng per mL⁻¹ hour⁻¹, and 0.058±0.004 ng per mL⁻¹ hour⁻¹, respectively.

### Bacterial Identification and Degradation Characteristics

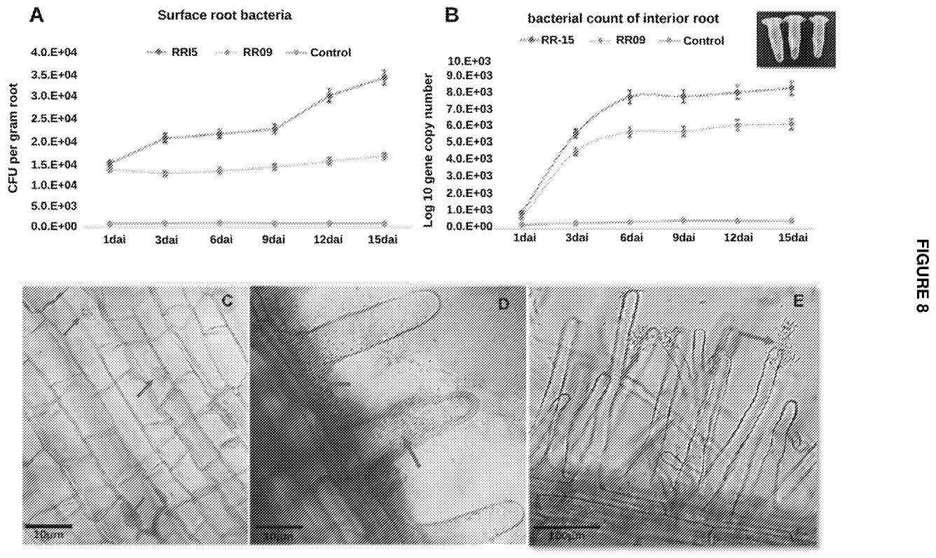

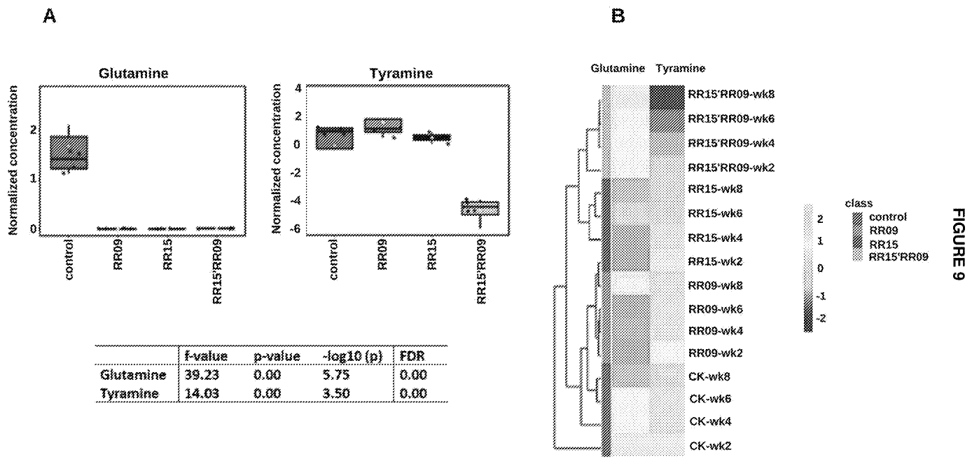

Part of the 16s rRNA gene of the five most promising bacterial isolates, based on rate of degradation of Gln and Tyr, were sequenced, revealing 99-100% homology to Pantoea sp. (RR15), Rhodococcus sp. (RR09), Bacillus sp. (RR08), Brevibacterium sp. (RR17), and Streptomyces sp. (RR12) (FIG. 6). These five species belong to three common phyla of soil bacteria, namely Firmicutes, Proteobacteria, and Actinobacteria. 16S rRNA sequence comparisons revealed that isolate RR15 shared 99% GC homology with P. vagans LEM67, while strain RR09 shared 99% GC homology to Rhodococcus sp. IN105 (FIG. 7).

### Colonisation of Bacteria in Roots

An assessment of the ability of the isolated rhizosphere bacterial strains RR15 and RR09 to colonise potato roots was undertaken by conventional colony-forming unit measurement, microscopic observation and quantitative PCR. The results showed that both isolates successfully colonised both internal and external surfaces of the root. When compared to RR09, strain RR15 colonised root hair surfaces and intercellular spaces at the elongation zone more quickly (FIG. 8A-B). In most cases, isolate RR15 was detected on the potato root differentiation zone and was visible on the surface of root hairs after inoculation (FIG. 8D), whereas isolate RR09 was observed to attach randomly along the basal region of the root hair surface while displaying no preferred site for external colonisation (FIG. 8C).

### Degradation of Key Compounds In Situ

The metabolomic profiles of control and bacterial-treated plants grown in pots under glasshouse conditions were evaluated using UPLC-MS/MS. In total, 39 low molecular weight compounds (16 amino acids and 23 other organic compounds) were determined in all samples. Both bacterial isolates RR15 and RR09 reduced target metabolites, either alone or in combination. The findings show that bacterial breakdown of Gln was significantly reduced after single or mixed inoculation of bacterial isolates, whereas Tyr degradation was slower, with only considerable loss observed after co-inoculation of bacterial isolates (FIG. 9A). While looking at the trend of Gln and Tyr in situ degradation over time, glutamine breakdown by bacterial isolate RR15 is visible as early as 2 weeks and is nearly undetectable after 8 weeks; nevertheless, even isolate RR09 degradation can be noticed as early as 2 weeks but somehow Gln levels start to rise after 8 weeks, whereas a decrease of Gln is delayed until 6 weeks in co-inoculations (FIG. 9B). Meanwhile, whether amended with single or mixed inoculations, breakdown of Tyr concentration occurs later after 4 weeks but the main difference is that, while RR15 and RR09 reduces Tyr levels for up to 8 weeks, the co-inoculation drops Tyr concentration up to the lowest detection limit as early as 4 weeks (FIG. 9B). Whilst in the control group, either Gin or Tyr levels remain high, with a level drop noted after the 8-week period (FIG. 9B).

When examining the various components found in the potato rhizosphere, it was observed that bacterial amended plants released a variety of amino acids and organic compounds in the soil exudates. Interestingly, succinic and lactic acid appear to be produced in large amounts in treated plants but not in control plants, whereas chlorogenic acid and caffeic acid appear to be quite high in control plants but progressively decrease in treated samples (Table 3).

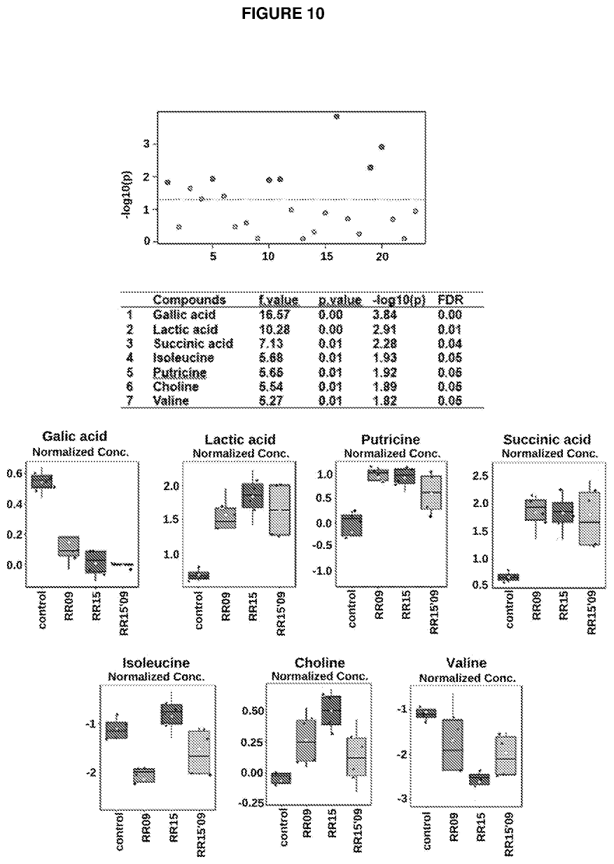

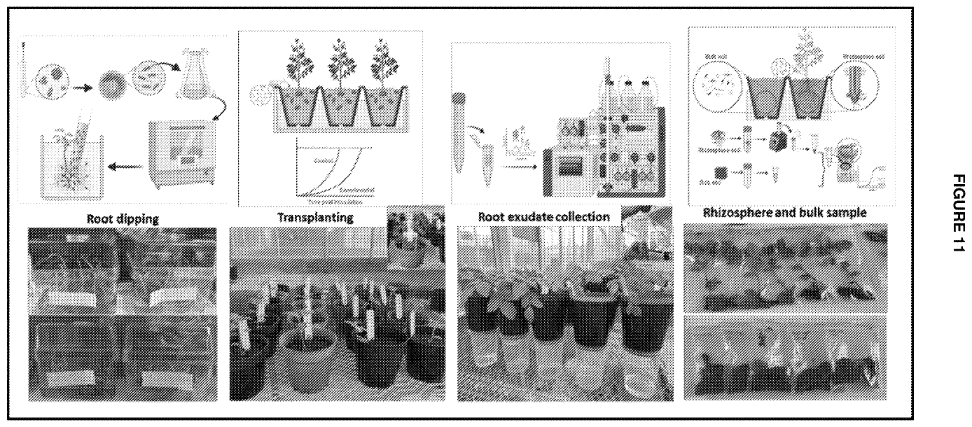

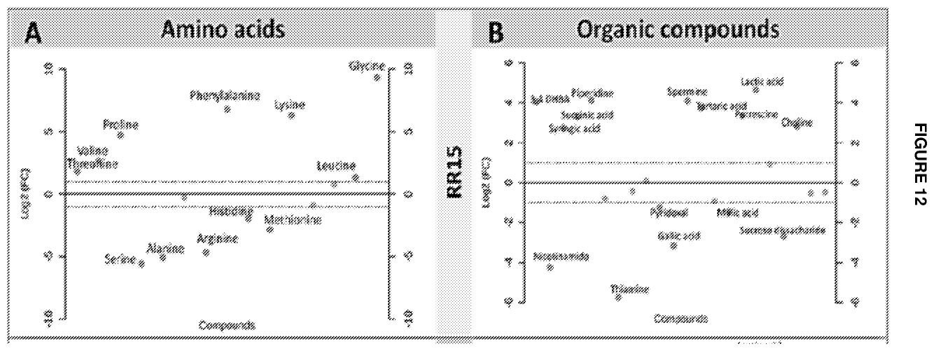

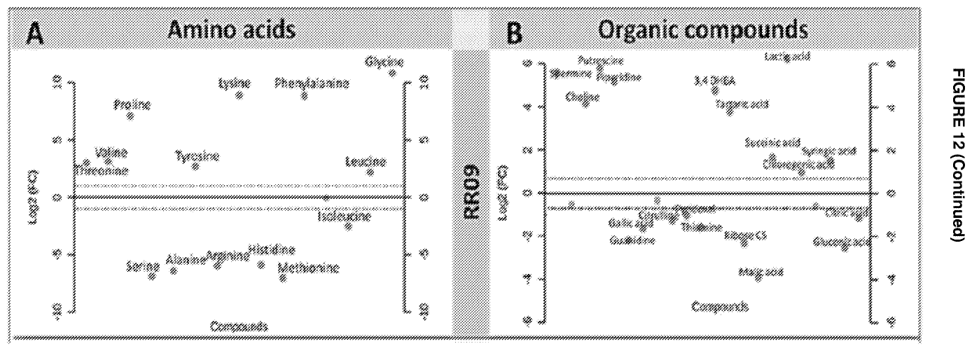

While the majority of the metabolome varies depending on treatments and time, several compounds were not detected in either the control or treated groups (Table 3) such as amino acids (Threonine, Serine, Alanine, Histidine, Methionine, Lysine) and organic compounds (Piperidine, Tartaric acid, Malic acid, Syringic acid, and Gluconic acid) as well as sugars (Ribose C5, Sucrose disaccharide and Glucose C6). However, there was a substantial fold difference between control and treatment groups for gallic acid, lactic acid, succinic acid, isoleucine, leucine, putrescine, choline, and valine (FIG. 10).

TABLE 3
Amino acid and organic components in the potato metabolome
quantified 8 weeks after bacterial inoculation

| Compound Type | Control | RR15 | RR09 | RR15*RR09 |
|---|---|---|---|---|
| Amino acids (AA) | exudate content ng/ml⁻¹ | | | |
| Threonine | ND | ND | ND | ND |
| Valine | 1.1 ± 0.5 | ND | 0.6 ± 0.1 | 0.10 ± 0.3 |
| Proline | 0.8 ± 0.5 | 0.7 ± 0.3 | 0.5 ± 0.2 | 0.4 ± 0.5 |
| Serine | ND | ND | ND | ND |
| Alanine | ND | ND | ND | ND |
| Tyrosine | 1.1 ± 0.2 | 0.6 ± 0.9 | 1.5 ± 0.4 | ND |
| Arginine | ND | ND | ND | ND |
| Phenylalanine | 8.8 ± 0.6 | 8.0 ± 0.3 | 7.8 ± 0.8 | 7.6 ± 0.4 |
| Histidine | ND | ND | ND | ND |
| Methionine | ND | ND | ND | ND |
| Lysine | ND | ND | ND | ND |
| Aspartic acid | ND | 12.9 ± 1.7 | ND | ND |
| Isoleucine | 0.9 ± 0.1 | 0.6 ± 0.5 | ND | 0.3 ± 0.3 |
| Leucine | 0.6 ± 0.2 | 0.7 ± 0.7 | ND | 0.5 ± 0.2 |
| Glycine | 6.2 ± 1.2 | 11.6 ± 1.3 | ND | 17.1 ± 0.1 |
| Total AA | 1.3 ± 0.6 | 2.4 ± 1.4 | 0.7 ± 0.4 | 1.7 ± 0.1 |
| Organic compounds (OC) | exudate content ng/ml⁻¹ | | | |
| Spermidine | 5.8 ± 1.2 | 44.7 ± 9.7 | 34.2 ± 4.5 | 8.9 ± 1.2 |
| Nicotinamide | 13.3 ± 2.0 | 11.5 ± 0.9 | 7.7 ± 1.6 | 7.2 ± 0.9 |
| Choline | 9.4 ± 2.0 | 13.9 ± 1.8 | 10.7 ± 3.1 | 10.3 ± 1.8 |
| Putrescine | 10.8 ± 2.9 | 36.8 ± 2.7 | 30.8 ± 4.4 | 15.8 ± 3.6 |
| Piperidine | ND | ND | ND | ND |
| Guanidine | ND | 42.4 ± 4.8 | 69.4 ± 5.8 | 30.7 ± 3.1 |
| Thiamine | 26.4 ± 5.2 | 33.9 ± 6.7 | ND | ND |
| Spermine | 63.2 ± 8.6 | 33.4 ± 5.2 | 42.6 ± 3.1 | 19.7 ± 2.2 |
| Citrulline | 5.2 ± 0.1 | 2.5 ± 0.1 | 11.2 ± 0.1 | ND |
| Gallic acid | 38.2 ± 4.4 | 13.4 ± 2.1 | 32.2 ± 4.1 | ND |
| Pyridoxal | 10.3 ± 2.4 | 4.1 ± 1.3 | 4.5 ± 1.6 | 2.2 ± 0.6 |
| 3,4 DHBA | 28.1 ± 2.3 | 11.1 ± 1.1 | 28.8 ± 2.2 | ND |
| Tartaric acid | ND | ND | ND | ND |
| Ribose C5 | ND | ND | ND | ND |
| Malic acid | ND | ND | ND | ND |
| Succinic acid | ND | 527.5 ± 136.1 | 486.7 ± 177.2 | 779.3 ± 192.5 |
| Lactic acid | ND | 527.1 ± 16.7 | 356.0 ± 13.3 | 274.4 ± 12.1 |
| Chlorogenic acid | 421.5 ± 9.9 | 57.6 ± 2.9 | 197.3 ± 15 | 39.5 ± 1.7 |
| Sucrose disaccharide | ND | ND | ND | ND |
| Syringic acid | ND | ND | ND | ND |
| Citric acid | 19.5 ± 13.5 | (—) | (—) | (—) |
| Gluconic acid | ND | ND | ND | ND |
| Caffeic acid | 98.8 ± 19.9 | 37.0 ± 4.9 | 47.6 ± 4.4 | 7.5 ± 2.8 |
| Glucose C6 | ND | ND | ND | ND |
| Total OC | 33.3 ± 4.2 | 58.2 ± 5.7 | 56.3 ± 4.8 | 49.6 ± 7.7 |

ND = below detection limit

### Discussion

In the present study, 12 culturable bacteria were isolated from the potato rhizosphere which were capable of growth on minimal media supplemented with Gln or Tyr as the sole carbon sources. Of these isolates, two (RR15 and RR09) showed the greatest capacity for rapid and complete degradation of Gln and Tyr. Morphological and molecular identification revealed that isolates RR15 and RR09 were members of the genera Pantoea and Rhodococcus, respectively.

Pantoea species are gram-negative, yellow-pigmented bacteria that belong to the Enterobacteriaceae family and include a broad range of species that have been isolated from diverse environments (Walterson & Stavrinides, 2015). Rhodococcus species are aerobic non-sporulating, non-motile, gram-positive members of the Nocardiaceae family (Johnson K B and Stockwell V O, 1998, Annual Rev. Phytopathol., 36:227-248). Both Pantoea and Rhodococcus species are predominant members of the soil community within the Proteobacteria and Actinobacteria, respectively, which have been documented to possess good capabilities for degrading metabolites and other organic molecules (Blaszak M et al., 2011, Water, air, and soil pollution, 220(1-4): 373-385; Zeng Q et al., 2016, pLoS ONE: 11(4): e0152894, https://doi.org/10.1371/journal.pone.0152894. Several Pantoea isolates have been developed into commercial biocontrol agents, with activity against pathogens by competitive niche exclusion, antibiotic production and an ability to induce plant systemic resistance (Johnson K B and Stockwell V O, 1998, supra; Johnson K B et al., 2000, Phytopathology, 90(11): 1285-1294).

Pantoea species also exhibit remarkable environmental versatility and adaptation, as well as a wide range of biosynthetic and biodegradative capacities. Some Rhodococcus strains have also been investigated for commercial purposes due to their unique capacity to degrade and/or convert refractory compounds, making them ideal for biocatalytic and bioremediation processes (Alvarez H, 2010, Biology of Rhodococcus. Springer Science & Business Media. pp. 231-256. Mendez-Volas, A. Microbes in applied research; current advances and challenges; proceedings. World Scientific. pp. 197-200.

Numerous reports documenting gram negative bacterial degradation of aromatics. For example, Pseudomonas putida has plasmid-encoded degradative nah genes encoding upper pathway enzymes involved in the conversion of naphthalene to salicylate, which is identical to Ralstonia sp. U2, nag genes containing upper pathway enzymes involved in the conversion of naphthalene to gentisate. We identified several gram-positive degrading bacteria in our study, however the gram-negative Pantoea isolate RR15 demonstrated the greatest efficacy at targeted metabolite degradation.

Root colonisation is one of the most crucial stages in the interaction between bacteria and their hosts. In this study, it is observed that within 24 hours, bacterial cell colonisation was often apparent on the root hair surfaces at the elongation zone. Results showed that Pantoea RR15 was capable of rapid and efficient root colonisation as early as 24 h after inoculation and achieving a maximum of 4×10⁴ CFU per gram root by day 15 This finding is comparable to that of Soluch R et al., 2021, Environ. Microbiol., 23(4): 2260-2273, who found that a Pantoea sp. colonised wheat roots rapidly across individual plants 5 h after inoculation and increased considerably in population size over the course of the experiment, eventually reaching a stable density of 5.1×10⁷ CFUs per gram root. According to Wheatley R M and Poole P S, 2018, FEMS Microbiol. Rev., 42:448-461, the rapid, consistent, and robust colonisation capacity of the Pantoea population is most likely mediated by pili and attachment proteins in combination with or in response to plant chemical signals.

The chemical composition of root exudates released into the rhizosphere altered following bacterial inoculation by week 8 of the glasshouse study. This finding is consistent with previous studies that also demonstrated changes in metabolite composition of the whole plant by specific soil microbes, indicating a bi-directional interaction between root and rhizosphere microbial populations (Curzi M J et al., 2008, Journal of Plant Interactions, 3: 163-173). In this study, out of the 39 compounds analysed, seven metabolites were differentially expressed in treated plants versus controls, with significant fold change observed for gallic acid, lactic acid, succinic acid, isoleucine, putrescine, choline, and valine. In addition to any direct effect of Pantoea RR15 root colonisation on these compounds, there could be indirect effects resulting from a close interaction of Pantoea RR15 with other competing microorganisms in the soil, which could affect the production of secondary metabolites, similar to the findings of Tyc O et al., 2017, Microb. Biotechnol., 10:910-925) who found that biotic interaction such as interspecific competition, particularly in the rhizosphere, triggers changes in metabolite production, as can be seen in Streptomyces, where interspecific interactions with other soil bacteria influence antimicrobial compound synthesis by both stimulating and inhibiting it.

In conclusion, this study demonstrated successful isolation and characterization of a rhizosphere bacterial isolate (Pantoea isol. RR15) that showed a significant affinity for the potato root rhizosphere and demonstrated a strong capacity to degrade pathogen stimulating compounds within potato root exudates. The targeted degradation of these critical metabolites that stimulate both pathogen germination and chemotaxis offers a novel approach to biological control of soil-borne diseases through manipulation of soil chemical ecology.

### Example 2

### Metabolic and Microbial Modulation Driven by Bacteria Degrading Phytochemicals: a Concept for Powdery Scab Disease Control

This aim of this study was to test the hypothesis that delivering bacterial degrader into the plant rhizosphere would elicit systemic changes in the exudation profile of the plant towards disease management. In this regard, the effects on management of powdery scab disease in potatoes was investigated.

### Materials and Methods

### Inoculum Preparation

In this study, bacterial isolates Pantoea sp. strain RR15 and Rhodococcus sp. strain RR09 identified in Example 1 were investigated. These specific isolates were chosen because they have the capacity to degrade critical chemicals involved in pathogen germination as well as having the ability to colonise and persist in potato roots in an artificial inoculation bioassay. The isolates were enriched at least six times, and a loopful of culture was streaked on LB agar plates. Single colonies were picked, re-streaked, and purified repeatedly by sub-culturing on fresh LB medium (NaCl 10 g L⁻¹, yeast extract 5 g L⁻¹, tryptone 5 g L⁻¹). Purified isolates were cultivated on LB agar, and bacterial stocks were cryopreserved in 50% (v/v) glycerol at −80 °C (Lami M J et al., 2020, J. Appl. Microbiol., 129(5): 1321-1336). An inoculum from each isolate was prepared from bacterial cultures grown in 50 mL LB broth and incubation at 23 °C on a rotary shaker (150 rpm) overnight. Cells were harvested by centrifugation at 6000×g for 10 min at 4 °C and the pellet was resuspended in sterile PBS buffer (10 mM NaH₂PO₄, 0.8% NaCl, pH 6.5) (Liao C H and Shollenberger L M, 2003, Letters in Applied Microbiology, 37:45-50). The final inoculum utilised for amendment and pot experiments was determined, and the OD₆₀₀ was adjusted to 0.5 using PBS buffer.

### Planting and Microbial Amendment

To determine the effects of the two bacterial isolates under investigation, a glasshouse experiment was established to identify and capture changes in metabolic profile, shifts in bacterial community structure in the rhizosphere, and their influence on plant development. All plants were sourced from a sterilised tissue cultured cuttings of Solanum tuberosum cv. Ranger Russet grown in tissue culture containers containing 20 ml of semi solid 1/2 strength Murashige and Skoog (MS) basal medium (Sigma-Aldrich, Darmstadt, Germany), containing 0.8% agar; pH 5.8 (Murashige T and Skoog F, 1962, Physiologia Plantarum, 15:473). After 3 weeks, individual seedlings (n=64) of about 3 cm height with 3-4-fully expanded leaves were uprooted and rinsed three times with sterile distilled water to remove all the attached semi-solid medium. Bacterial application was done prior to transplanting by dipping the seedling roots in an inoculum suspension of each isolate (containing ~10⁸ CFU per mL⁻¹) for 5 hours, whereas control seedlings roots were dipped in sterile distilled water. Following bacterial dipping, seedlings were transferred into pots (17 cm×19 cm, with drainage holes in the bottom) filled with 2.5 L of field (ferrosol) and potting mix soil (1:1:8 mix of peat, coarse sand, and composted pine bark) with seedlings then lightly watered to aid establishment. A total of 160 pots were used in this study, divided into 2 groups: (1) pots with plants (rhizosphere soil), and (2) pots without plants (bulk soil), with each receiving the following treatments: 1) ddH₂0 as a negative control; 2) Pantoea sp. strain RR15; 3) Rhodococcus sp. strain RR09; and 4) Dual bacterial inoculation of RR15+RR09 (DBI). Each treatment had five replications that were sequentially assessed at four different time periods (2, 4, 6 and 8 weeks). The experiment was carried out in the glasshouse maintained at 20±3 °C and the additional illumination providing a photoperiod of 16-h light/8-h dark. Pots were watered 2-3 times per week to maintain a moist soil suitable for plant growth. At each sample period, a 100 mL leachate (containing root exudates) was carefully collected from each pot (with plants and without plants) that received the bacterial treatments RR15, RR09, co-inoculation, and the control. After the final collection of leachates, three soil core samples (15 cm depth) were collected and pooled as bulk soil samples taken from treated pots (pot without plants) whereas rhizosphere soil sample was drawn from treated pots (pot with plants). Each soil sample was collected in triplicate. The relative growth of all plants were then assessed. Each plant was harvested, roots were washed several times to remove organic material, and measured for root and shoot length, root and shoot fresh weights, and then dried at 60 °C for 48 hours before re-weighing to dry matter content.

### Analysis of Pot Leachates

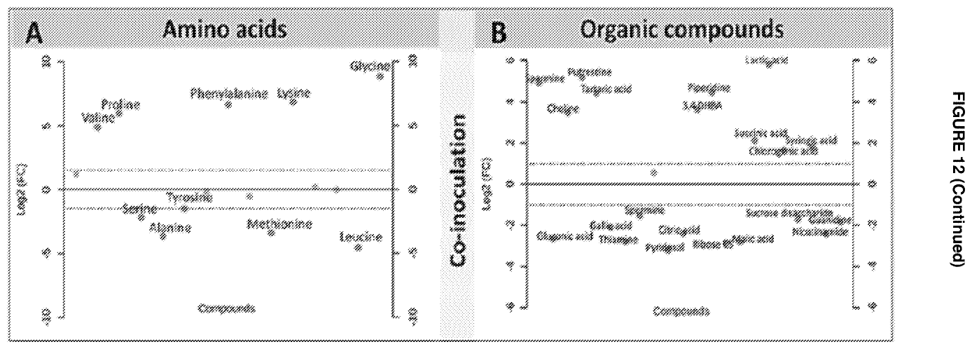

Root exudates were collected at prescribed times over the study period. Exudates were collected by slowly pouring sterile water over the complete plant-soil system soil surface until 100 ml of leachate was collected from beneath the pot (FIG. 11). The leachate was separated into two 50 mL portions, one serving as an insurance stock and the other for metabolomic analysis. The metabolomic sample was placed into a 50 mL falcon tube and centrifuged for 5 minutes at 7000×g. After that, the supernatant was collected and syringe filtered through a 0.22 μm filter (Merck Millipore, Darmstadt, Germany), and all samples were promptly frozen at −20 °C for storage (Williams A et al., 2021, Soil Biology and Biochemistry, 161). To determine the root exudation composition, frozen root exudate samples were thawed and 1 mL of each were transferred into HPLC glass vials, with root exudate extract identified and quantified using a high-precision ultra-high-performance LC-tandem MS technique with an additional internal standard, as described by Gika H G et al., 2012, J. Chromatogr., 1259:121-127. A Waters Acquity H-Class UPLC was used to examine potato metabolites (Waters, Millford, MA). The solvents used in the mobile phase were solvent A (acetonitrile/water, 95/5 (v/v), 0.1% formic acid, and 0.075% NH₄OH), and solvent B (acetonitrile/water, 2/98 (v/v), 0.2% formic acid, and 0.1% NH₄OH). The gradient program started with a 4-minute isocratic step at 100% mobile phase A, then progressed to 28% mobile phase B, which was held for 21 minutes, and finally to 60% mobile phase B over 5 minutes, followed by a 12 minutes re-equilibration to the initial condition. Between injections, the injection system was subjected to two cycles of weak and strong solvent washing. The column eluent was directed to the mass spectrometer with a 10 μL injection volume. Metabolite detection was accomplished by utilizing selective ion monitoring and an electrospray ionisation source that worked in both positive and negative ion modes. The electrospray settings were as follows: capillary voltage of 2.5 or 3 kV, cone and desolvation temperatures of 150 and 400 °C, respectively, with a desolvation gas flow of 950 L/h and a cone flow of 100 L/h. For each analyte, the cone voltage was optimised by analysing standard compounds as both pure standards and injected into root exudates samples, the identification of metabolites was verified by the detection of the predicted [M+H]+ or [MH] molecules at recognised retention indices. The MRM transition responses were averaged for each analyte, and peak identification and quantification were performed using MassLynx™ software (Waters Corporation, Milford, MA). Basic test performance characteristics were evaluated using standard laboratory procedures (sensitivity, precision, accuracy, and linearity). In order to analyse the difference and fold change between treatments, raw metabolite data was log 10 converted to meet the requirements of statistical models and to normalise distribution among putative metabolites. A cluster heatmap based on K-means clustering of mean metabolite intensity for each species was generated to visualise the influence of treatments on metabolite profiles. To determine how much metabolites differed between the control and treated groups, univariate analyses (t-tests) were performed on log 10 transformed data for each metabolite, comparing control plants to T1 (inoculated with isolate RR15), T2 (inoculated with isolate RR09), and T3 (inoculated with both isolates RR15 and RR09) plants (co-inoculation). The Benjamini-Hochberg false discovery rate (FDR) adjustment was used to adjust p-values for multiple comparisons. To compare metabolite features that change consistently (i.e. up-regulated or down-regulated) between groups a fold change (FC) analysis was employed to obtain putative identities from all three binary comparisons (Control vs T1_+RR15, Control vs T2_+RR09, Control vs T3_co-inoculation) by selecting significant metabolites (−log 10 p-value >5) that also had a log 2 fold-change greater than 2.0 or below 2.0). These analyses were performed using the R package MetaboAnalystR version 5.0 (https://github.com/xia-lab/MetaboAnalystR) (Bijlsma S et al., 2006, Anal. Chem., 78(2): 567-574).

### Bacterial Monitoring and Recovery

### DNA Extraction

The rhizosphere soil sample was taken from soil fractions with ~1 mm of soil attached to the roots. Roots were lightly rinsed and transferred into 50 mL falcon tubes containing sterile distilled water and vortexed for 20 mins in falcon tubes. The bulk soil sample (5 g pooled sample) was sieved a 2 mm mesh and agitated using ultrasonic cleaner for 20 mins as for the rhizosphere soil. The collected soil slurries were centrifuged for 15 minutes at 13,000×g. The supernatant was discarded, and DNA was isolated from the remaining centrifuged soil fraction. The collected soil fraction was pre-homogenized prior to DNA extraction by bead beating for 1 min in 5.5 m/s speed using the Fast Prep-24 Classic bead beating grinder and lysis system system (MP Biomedicals, OH, USA). From each 250 mg rhizosphere and bulk soil sample, genomic DNA was extracted using the dNeasy Power Soil Pro Kit (Qiagen, USA) according to the manufacturer's instructions. DNA quality and concentration were measured using a Qubit® 2.0 Fluorometer (Life technologies, Darmstadt, Germany), and diluted as a stock concentration to 5 ng total gDNA/μL using sterile water for subsequent analyses. In each sample, a gBlock fragment with a concentration of 10 ng/μL was used as a DNA standard in a quantitative PCR assay (see below).

Quantitative PCR (qPCR) Analyses

Bacterial inoculant recovery from the rhizosphere and in bulk was examined throughout the eight weeks of potato growth using the Rotor Gene-Q Real-Time PCR System (Qiagen, Hilden, Germany), utilising three biological and three technical replicates for qPCR studies to quantify the individual genes of each isolated DNA. Two pairs of specific primers for the two bacterial inoculants amended were chosen to detect the copy numbers of 16S rRNA gene in V3-V4 region variable regions. These primers were RR15-2F (5′ ACGGCCGCAAGGTTAAA-3′) (SEQ ID NO: 3) and RR15-2F (5′-GCTGGATGTCAAGAGTAGGTAAG-3′) (SEQ ID NO: 4), RR09-1F (5′-CGGAATCACTGGGCGTAAA-3′) (SEQ ID NO: 6) and RR09-1R (5′-GTTCCACCAACCTCTACCATAC-3′) (SEQ ID NO: 7).

The qPCR amplification was carried out in a 20 μL final volume containing of 2 μL of each 5 ng/μL of total gDNA, 0.5 μL of each 10 μM forward and reverse specific bacterial primers and specific probes (RR15-2prb 5′-ACAAGCGGTGGAGCATGTGGTTTA-3′-SEQ ID NO: 5; RR09-1prb 5′-CGGAATCACTGGGCGTAAA-3′-SEQ ID NO: 8), 10 UL of 1× SensiFAST™ Probe No-ROX Kit (Bioline Meridian BioScience, USA) and 5 UL sterile DNA-free water. The reaction was carried out following thermocycling conditions comprising an initial denaturation for one cycle at 95 °C for 30 sec, followed by amplification 40 times at 95 °C for 10 seconds, 59 °C for 20 seconds, and 72 °C for 20 seconds. Three technical replicates were performed on each rhizosphere or bulk DNA sample, and to normalise qPCR analysis the marine bacterium Pseudoaltemoranas prydzensis was employed as an internal control (Bowman J P, 1998, supra; Kamilova F et al., 2006, Mol. Plant-Microbe Interact., 19:250-256). A standard curve was established using 10-fold serial dilutions ranging from 10⁸-10² copies per reaction of a gBlock fragment containing the targeted DNA PCR fragment of 16S rRNA gene of bacteria. All target genes and runs were reported to be 99% efficient, with R² values above 0.99%. The target gene's copy number was determined using the calibration curve of gBlock fragments. All data analysis was carried out using the Rotor-Gene Q software package (Qiagen, Hilden, Germany).

### Soil Community Bacterial Profiling

The bacterial microbiome from the rhizosphere and bulk soil of an inoculated potato cultivated in a glasshouse was examined. Total genomic DNA was extracted from 0.25 g of rhizosphere or bulk soil samples using the dNeasy Power Soil Pro Kit (Qiagen, USA) according to manufacturer's guidelines. The amount of DNA extracted was measured using a Qubit® 2.0 Fluorometer (Life technologies, Darmstadt, Germany) to obtain the standard DNA concentration (~20 ng) required by the sequencing service. Segments of the 16S rRNA gene were then amplified using primer pairs of 341F (5′-CCTAYGGGRBGCASCAG-3′-SEQ ID NO: 9) and 806R2 (5′-GGACTACNNGGGTATCTAAT-3′-SEQ ID NO: 10) targeting the V3-V4 region of bacteria. The amplicon sequencing procedures were carried out on an Illumina belfastq 2.20.0.422 pipeline at the Australian Genome Research Facility (AGRF, Melbourne) according to quality standards (Caporaso J G et al., 2010, Nat. Methods, 7(5): 335-336; Caporaso J G et al., 2011, PNAS USA, 8:7). Using an in-house Illumina MiSeq sequence workflow, 300 bp paired-end sequences were combined and initially denoised to sequences that contained sequencing errors. Diversity profiling analysis was performed with the bioinformatics tool QIIME2 2019.7 (Bolyen E et al., 2019, Nat. Biotechnol., 37(8): 852-857). The demultiplexed raw read are primer trimmed and quality filtered using the cutadapt plugin followed by denoising with q2-DADA2 (Callahan B J et al., 2016, Nat. Methods, 13(7): 581-583). Taxonomy was assigned to ASVs using the q2-feature-classifier (Bokulich N et al., 2018, mSystems, 20(3): 6).

### Evaluation of Plant Growth Parameters

Following bacterial amendment in a controlled environment, a completely randomised design with five replications was used to distribute the treatments. To assess growth parameters, five plants were carefully uprooted and washed via running tap water to remove adhering soil debris, aerial parts and roots were separated to analyse plant development indicators. Six quantitative plant growth characteristics were determined: plant height (cm), root length (cm), shoot length (cm), fresh mass of root and shoot (g), root-shoot ratio, and percent dry matter content. For dry weight measurements, biomass was dried to a constant weight in an oven at 60 °C for 48 hours. The plant growth parameter data were presented as means±standard deviations (SD). Quantitative differences were evaluated by one-way analysis of variance. Duncan's multiple-range test was performed to separate the means. A significance threshold of p<0.05 was applied. Statistical analyses were performed using IBM® SPSS® Statistics 28.0.0.0 (SPSS Inc., USA).

### Results

### Impact of Bacterial Inoculants on Root Exudation Profile

To better understand the specific alterations in plant root exudates from potato plants treated with the bacterial inoculants under investigation, high-resolution mass spectrometry was performed. UPLC MS/MS analysis of major root exudate components revealed that the bacterially treated plants significantly altered the quantity of amino acids and organic compounds released in the rhizosphere. A total of 14 amino acids and 21 organic compounds were significantly altered in rhizosphere following additional of bacterial treatment (two-fold change cut-off between bacteria-treated and the non-treated controls, P<0.05, one-way ANOVA followed by multiple comparisons with FDR correction).

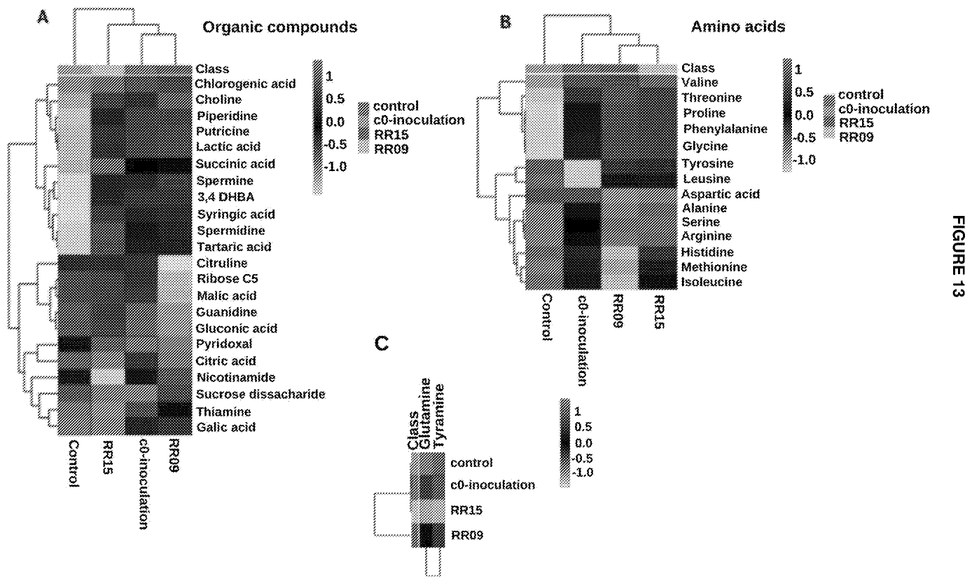

The log 2 fold change analysis demonstrated that plants treated with a single bacterial inoculant (Pantoea sp. strain RR15 or Rhodococcus sp. strain RR09), or with a co-inoculation of these strains, had significantly altered metabolite profiles compared to non-treated controls (p≤0.05) (FIG. 12). The fold change differences in amino acids and organic compounds between treatment groups and control groups revealed a wide range of upregulation and downregulation of these components. Metabolites from bacterially treated groups were found to have similar metabolic characteristics in terms of their regulation. For example, the amino acids glycine, phenylalanine, lysine, proline, valine, and threonine were shown to be significantly increased in all bacteria-treated plants; however, tyrosine was only increased in RR09-treated plants. Meanwhile, all treatments reduced histidine, methionine, arginine, alanine, and serine, with the exception of leucine, which was upregulated in plants treated with a single bacterial inoculant and downregulated in plants with a co-inoculation of strains (FIG. 12A).

Fold changes in organic compounds revealed a wide range of upregulated and downregulated metabolite components. Nine organic compounds showed a significant increase in all bacterial-treated plants, including lactic acid, spermine, tartaric acid, putrescine, choline, piperidine, 3,4 DHBA, succinic acid, and syringic acid, whereas pyridoxal, malic acid, gallic acid, and thiamine were all downregulated. Other chemicals, such as nicotinamide and sucrose disaccharide, were reduced only in RR15-treated and co-inoculated plants, while ribose C5, gluconic acid, guanidine, and citric acid were all significantly reduced only in RR09-treated and co-inoculated plants (FIG. 12B).

Based on these results, bacterial treatment impacted upregulation and downregulation of amino acids and organic compounds, resulting in differences in absolute fold change values of metabolites relative to controls. The degree of amino acid and organic compound variation depended on whether a single or co-inoculated bacterial treatment was utilised. Bacterial application resulted in an increase or decrease in the level of some metabolites. Pantoea sp. RR15 and/or Rhodococcus sp. RR09 applications, for example, increased the level of some amino acids by 1.7 to 10.8 log 2 FC and some organic compounds by 1.4 to 9.1 log 2 FC. Co-inoculation, on the other hand, elevated some amino acids by only 1.9 to 10.82 log 2 FC and organic compounds by 1.2 to 5.7 log 2 FC (FIG. 12).

Alternatively, utilising heatmap analysis showed clustering of metabolites based on the quantity of compounds found in the rhizosphere over time, with the overall exudation patterns from control plants were found to be distinctly different to those of treated plants (FIG. 13). Within treatments, organic compound release was closely comparable between RR09 and the co-inoculated group, however RR15-treated plants were more variable than the other two groups. In contrast to organic compounds, components of metabolites present in the rhizosphere were similar in both single treatment groups compared to the co-inoculation treatment (FIG. 13B). It should be noted that bacterial treatment of plants reduced the levels of stimulating phytochemicals like glutamine, tyramine, serine and nicotinamide. Furthermore, compounds inhibitory to zoospore taxis, spermine and choline, were found to be promoted with combined treatment application resulting in higher non-stimulatory compound production than when applied alone. Remarkably, important compounds which have previously been found to mediate germination of powdery scab resting spores (namely glutamine and tyramine), were all suppressed by inoculation with a single bacteria, or with a combination of bacteria, although the RR15-treatment alone reduced these stimulants the most (FIG. 13C).

### Bacterial Recovery and Influence on Community Composition

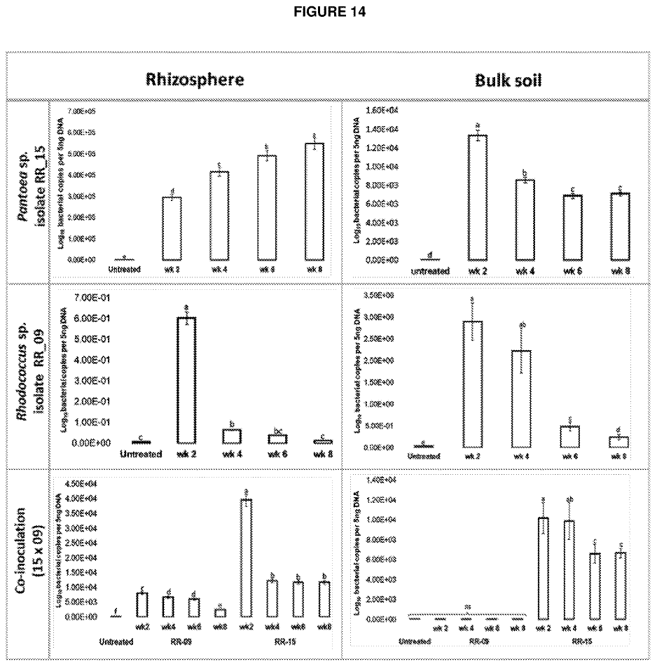

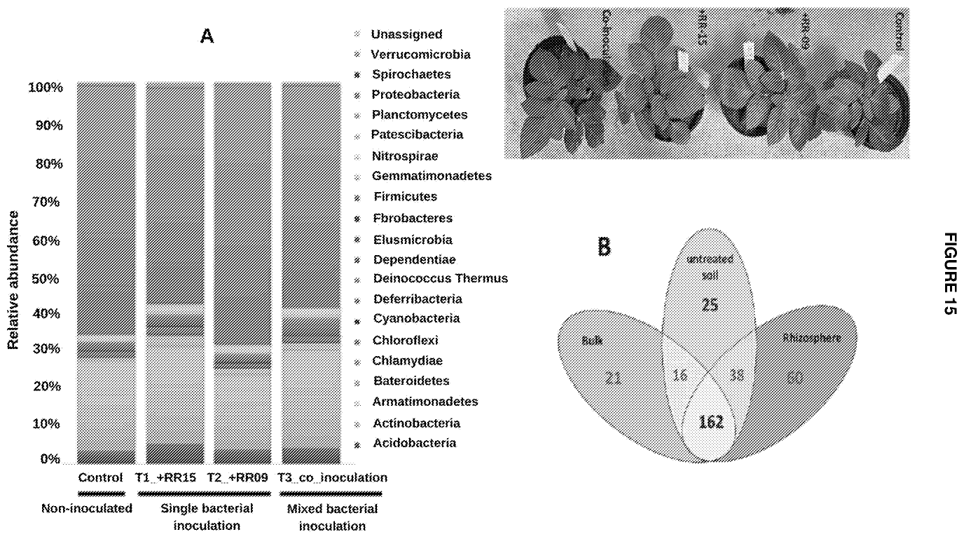

To evaluate the recovery of introduced bacteria, potato rhizospheric and bulk soil samples were obtained from bacterial treated plants. Among the bacterial isolates tested, Pantoea sp. strain RR15 displays better success in re-establishment than Rhodococcus sp. strain RR09. qPCR results demonstrate that Pantoea strain RR15 can efficiently re-establish in the rhizosphere and bulk soil, and that the activity and cell number of the isolate grows over time when roots (5.5×10⁵ log₁₀ DNA copies/5 ng total DNA) are present, and that it can sustain its establishment and activity even when roots are absent (7.11×10³ log₁₀ DNA copies/5 ng total DNA). Rhodococcus strain RR09, on the other hand, establishes itself in the rhizosphere as early as 2 weeks, although its population drops later, with just 1.34×10⁻² log₁₀ DNA copies/5 ng total DNA in the rhizosphere and around 2.40×10⁻¹ log₁₀ DNA copies/5 ng total DNA in bulk soil. Furthermore, when bacterial isolates were combined, re-establishment was less successful than when they were introduced singly, but it was still possible to observe both bacteria established in the rhizosphere displaying adequate cell density in week 2. The establishment of Pantoea strain RR15 within the rhizosphere seems to likely to persist longer than RR09. In contrast to their activity in the rhizosphere, both bacterial isolates exhibited a reduction in population in bulk soil, but Pantoea strain RR15 was able to maintain a DNA level of up to 6.7×10³ log₁₀ DNA copies/5 ng total DNA even after week 8, whilst Rhodococcus strain RR09 was near undetectable as early as 2 weeks (FIG. 14).

The influence of bacterial amendment into the rhizosphere on the composition of the soil bacterial community in potato-cultivated soil was investigated using the Illumina MiSeq technology and 16S rRNA gene amplicon sequences. In this study, a total of 5735 detected OTUs across samples, 28 phyla and 817 genera, were identified. The relative abundance of the soil bacterial community in non-treated and treated soil revealed that two bacterial phyla predominated in the rhizosphere, with Proteobacteria accounting for 92-95% and Actinobacteria accounting for 27-42%. In addition, other phyla such as Acidobacteria (4-9%), Firmicutes (3-7%), Chloroflexi (1-3%), Bacteroidetes (1-5%), Verrumicrobia (1-2%) and Gemmatimonadetes (2-4%) were also found encompassing the rhizosphere community (FIG. 15A). Noticeably, bacterial application promoted presence of other bacterial communities such as Patescibacteria (0.09%) and Defferibacteres (0.06%). Among the bacterial treatments, RR15-treated and co-inoculated treated soil exhibit substantial changes in bacterial make up, but RR09-treated and non-treated soil exhibit no obvious shift in phylum composition (FIG. 15A). Distinctively, RR15-treated soil promotes the population of Proteobacteria (9.53%), Actinobacteria (4.22%), Acidobacteria (0.92%), Bacteroidetes (0.52%), Chloroflexi (0.37%), Gemmatimonadetes (0.37%), and Verrumicrobia (0.23%), whereas combined bacterial inoculation promotes an increase of Firmicutes by about 0.73%. Furthermore, when comparing the impact of bacterial change at the genus level between the bulk and rhizosphere and non-treated samples (FIG. 15B), a total of 162 genera were found in common across samples, with 16 genera common between control and bulk and 38 genera common between control and rhizosphere.

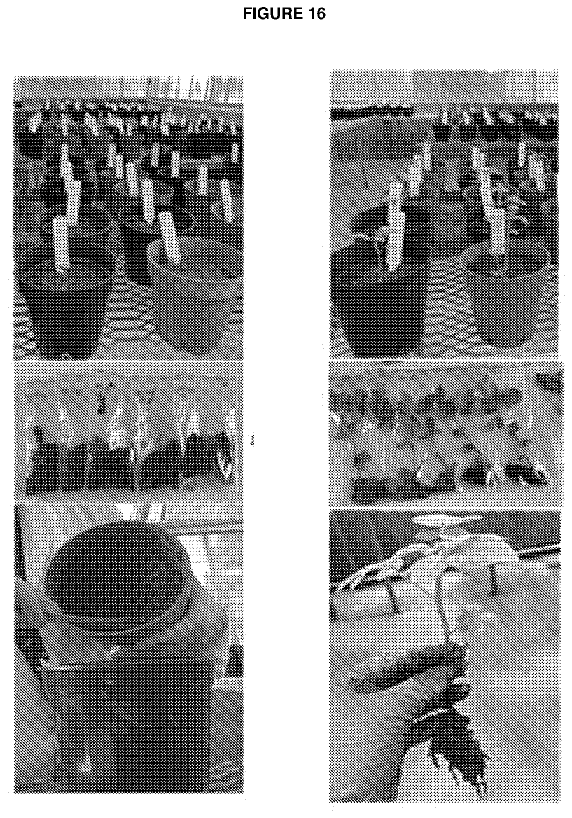

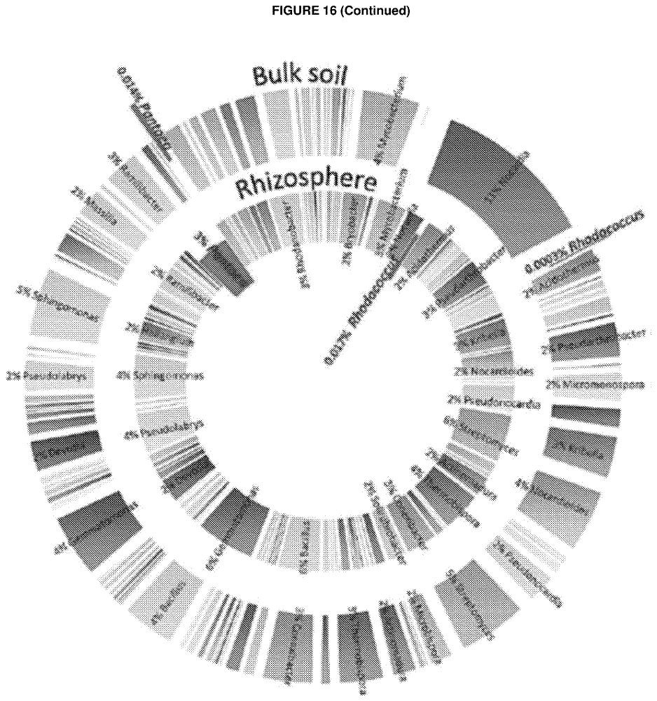

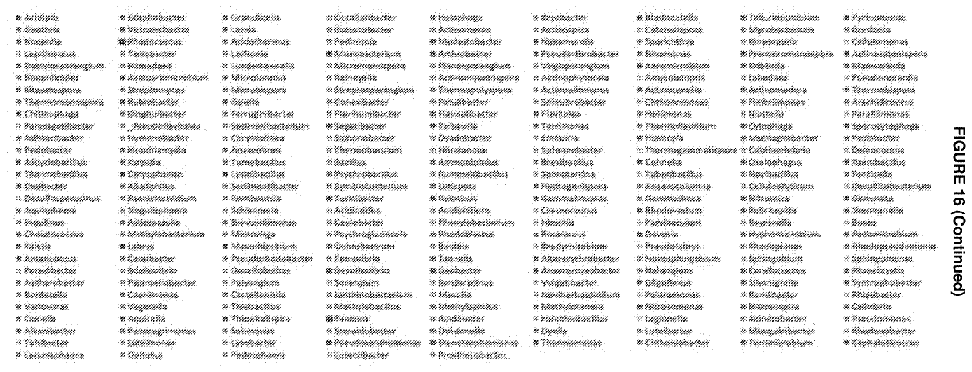

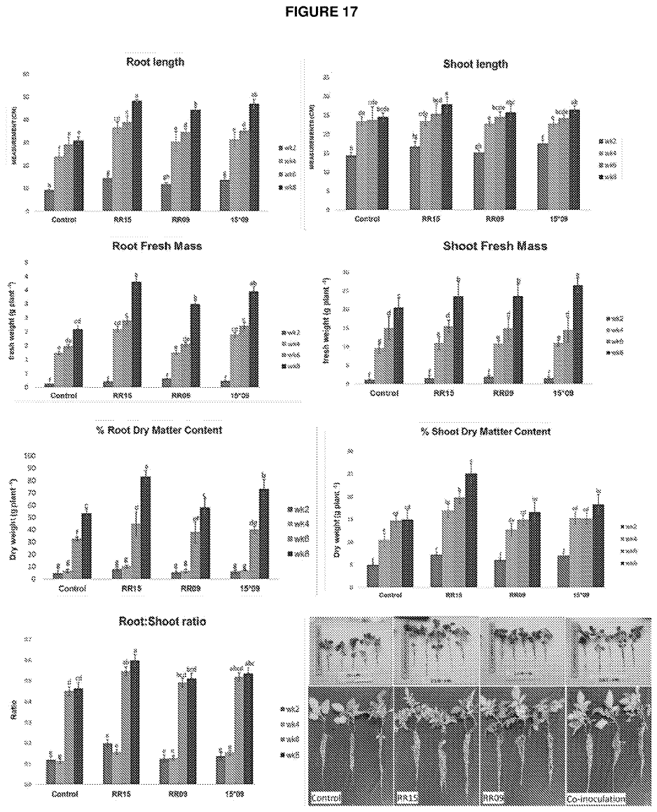

It is worth noting that five bacterial genera were discovered solely in bulk soil, whereas 22 bacterial genera were found exclusively in the rhizosphere, suggesting that the rhizosphere had a greater effect on bacterial application. On the other hand, when examining the differential composition between the bulk and rhizosphere soil communities, it is apparent that they differ significantly, with the bulk community dominated mostly by the bacterial species Nocardia (11%), followed by 5% communities of Streptomyces, Sphingomonas, 4% communities of Mycobacterium, Nocardioides, Bacillus, and Gemmatomonas, 3% communities of Kribella, Thermobispora, Conexibacter and Ramilibacter, and 2% communities of Rhodanobacter, Acidothermus, Pseudarthrobacter, Micromonospora, Pseudonocardia, Microbispora, Actinomadura, Devosia, Pseudolabrys and Massilia, whereas rhizosphere population accounts 6% Gemmatomonas, Bacillus and Streptomyces, 4% communities of Thermobispora, Sphingomonas, Pseudolabrys, 3% communities of Pantoea, Mycobacterium, Pseudarthrobacter, Kribella, Conexibacter and Rhodanobacter, and 2% communities of Ramilibacter, Bryobacter, Nocardia, Acidothermus, Micromonospora, Nocardioides, Pseudonocardia, Actinomadura, Solirubrobacter, Devosia and Haliangium. However, while changes of community in bulk and rhizosphere is accompanied by proliferation of a diverse range of soil bacteria, it is worth noting that the relative abundance of introduced inoculant Pantoea strain RR15 was able to encompass roughly 3% in the rhizosphere and 0.14% in bulk bacterial community, whereas Rhodococcus strain RR09 made up 0.02% and 0.0003% of rhizosphere and bulk community, respectively (FIG. 16).

### Effect of Bacterial Inoculation on Plant Growth Traits

The effect of single or co-inoculated bacterial application resulted in significant improvements in plant growth indices relative to the control (P≤0.05) (FIG. 17). In the presence of bacteria, root development showed remarkable effects, with Pantoea strain RR15-treated roots displaying a significant increase in root growth (with an average of 35±2.8 cm), followed by the co-inoculation group (with an average of 32±1.3), and the Rhodococcus strain RR09 treatment group (with an average of 30±2.1). This was in contrast to the control (non-treatment) group whose root growth was on average 23±0.9 cm.

Contrary to root growth, bacterial application had little discernible effect on shoot length with small but significant increases noted only for the bacterial co-inoculation at 2 weeks growth and for inoculation with the Pantoea strain RR15 at 2 and 8 weeks growth. Other growth indices, such as fresh mass and dry weight of root, as well as root: shoot ratio, were greatly influenced by the bacterial treatment. In the presence of bacteria, root weight was roughly 1-2-fold greater for treated plants than for controls, with RR15-treatment showing at least 1-fold higher than other bacterial applications. Nonetheless, all plants treated with isolates RR15 or RR09, as well as co-inoculation with both bacteria, exhibited a favourable response compared to the control (FIG. 17).

### Discussion

Numerous studies have discovered that bacteria established in the rhizosphere influence plant root exudation (Matilla M A et al., 2010, Environ. Microbiol. Rep., 2(3): 381-388). Early research suggests that various bacterial species have unique impacts on the quantity and composition of plant root exudates, and that rhizobacterial inoculation, for example, can quantitatively modify root exudation (Fransson P M A and Johansson E M, 2010, FEMS Microbiol. Ecol., 71(2): 186-189). Additionally, it may influence a plethora of other soil bacteria within the rhizosphere fraction, resulting in greater plant resilience to a range of abiotic and biotic stresses as well as plant growth promotion.

This work in this study provides evidence that application of particular bacterial isolates alters potato root exudation of amino acids and low molecular weight secondary metabolites, and facilitates a shift in the microbial community while simultaneously stimulating enhanced plant development. According to the findings of this study, root exudates emitted from bacterially treated plants were invariably higher than those of non-treated control plants. Quantitative variations in exudate profiles resulting from single and mixed bacterial inoculation indicate a specific pattern of concentration gradient shift that might increase or decrease significantly. Moreover, when we looked at the ability of bacterial inoculants in degrading the critical compounds glutamine and tyramine, which are involved in pathogen resting spore germination, we found a significant reduction by bacterial application, either alone or in combination, with Pantoea sp. strain RR15 showing the greatest effect on stimulant compound suppression. Other stimulants, such as serine, nicotinamide, and aspartic acid, were decreased, whereas chemicals that were identified to be non-stimulatory to pathogen germination, such as spermine and choline, increased significantly in abundance. This finding shows that Pantoea sp. strain RR15 may be comparable to other Pantoea soil isolates reported with high degrading capabilities, such as the capacity to breakdown herbicide from agricultural run-off (Pileggi M et al., 2012, Chemosphere, 86(11): 1127-1132) and remove heavy metals from waste water (Ozdemir G, 2004, Chemical Engineering Journal, 102(3): 249-253). Other Pantoea isolates, on the other hand, have been shown to be capable of producing compounds like glycolipid biosurfactant, resulting in emulsification and subsequent biodegradation of hazardous hydrocarbons (Vasileva-Tonkova E and Gesheva V, 2007, Curr. Microbiol., 54:136-141). To provide an understanding of the extent of bacterial survival in the rhizosphere over time, our data demonstrates that Pantoea strain RR15 persists longer in the soil. The results demonstrate that the bacterial densities of Pantoea strain RR15 are within the range of the typical bacterial densities often found in the rhizosphere, implying that RR15 is capable of competently establishing within the potato rhizosphere.

The high-throughput sequencing results give an insight into the true scale of the influence of bacterial introduction in restructuring of the potato rhizosphere microbiome. The findings in this study indicate that bacterial application of both the Pantoea sp strain RR15 and the Rhodococcus sp. strain RR09 affects the structure and composition of bacterial communities in rhizosphere and in bulk. Rhizosphere and bulk samples showed relatively consistent microbial composition with the exception of Nocardia which showed a great fluctuation of population between rhizosphere and bulk community, but other genera comprised 5-6% of the entire community (such as Bacillus, Gemmatomonas, Streptomyces, Sphingomonas, Mycobacterium) which represents a significant portion of the stable community. In particular, we observed that roughly 0.1-0.5% of additional bacterial taxa emerge, indicating a viable microbial presence exclusively in the rhizosphere as compared to bulk and untreated samples.

The increase of amino acids and other organic compounds in Pantoea-RR15 treated plants may have attracted more specific microorganisms that stimulate the general microbial population and drive the observed increase in microorganisms in rhizosphere substrates. The higher abundance of beneficial genera in Pantoea strain RR15-treated soil (like Bacillus and Streptomyces for example which are widely used in agriculture as plant growth-promoting and disease-suppressing agents), strongly suggests that Pantoea RR15 isolates play an important role in transmitting beneficial assemblage of bacteria in the plant microbiome and therefore an advantage to plant stress regulation and even for growth benefits. Meanwhile, all bacterially applied samples exhibited considerable growth in biometric parameters of potato development (root and shoot length, fresh mass and dry weight, root: shoot ratio), showing that the treated plants had better vigour and growth activity than the controls. When compared to other bacterial treatments, Pantoea RR15 application revealed improved growth characteristics, particularly in roots.

### Conclusion

This is the first study to look at the exudation characteristics of potato roots in response to bacterial application. Overall, the chemical changes observed in the rhizosphere, particularly the degradation of compounds associated with S. subterranea resting spore germination and zoospore chemotaxis, may reduce the numbers of released zoospores and their successful attraction to the potato root system resulting in less disease. Furthermore, the ability of Pantoea sp. strain RR15 to operate as a bioinoculant may provide a benefit in terms of increased biological activity, resulting in a pool of potential inhibitory chemicals and other microbiota that will aid in the control of Spongospora subterranea infection.

### Example 3

### Metabolomics to Elucidate the Role of Strain Pantoea sp. RR15 During Disease and Metabolic Transitions Following Spongospora subterranea Infection

In this study, targeted metabolomics using ultra-performance liquid chromatography coupled with tandem mass spectrometry (UPLC-MS/MS) was employed to investigate the metabolic changes associated with the establishment of Pantoea sp. RR15 to the potato rhizosphere following disease infection and to infer their role in plant growth promotion.

### Materials and Methods

### Bacterial Cultivation

Pantoea sp. strain RR15, which as shown in Examples 1 and 2 can successfully colonise potato roots and boost plant development, was employed for this study. A 1 mL bacterial suspension derived from a 50% glycerol inoculum stock was grown in a 250 ml flask containing 100 mL of freshly sterilised Luria Bertani broth (NaCl 10 g L⁻¹, yeast extract 5 g L⁻¹, tryptone 10 g L⁻¹). The culture was incubated at 23 °C with 150 rpm agitation for approximately 24 h until it attained an optical density of 1.0 (λ=600 nm). To prepare the cell suspension for inoculation, the culture medium was centrifuged at 3000 rpm for 10 minutes at room temperature to obtain a bacterial pellet, which was resuspended in 0.85% saline solution adjusted to OD₆₀₀=1.0. Spongospora subterranea inoculum was prepared by excising lesions from infected potato tubers, air-drying excised material at 25 °C, and then grinding and sieving through a 35 μm mesh to produce a powder inoculum, which was stored at 4 °C until use.

### Plant Material and Inoculation

Tissue cultured cuttings of susceptible (Solanum tuberosum cv. lwa) and resistant (Solanum tuberosum cv. Gladiator) potato cultivars were micro propagated on potato multiplication (PM) medium (MS salts and vitamins, 30 g/L of sucrose, 40 mg/L of ascorbic acid, 500 mg/L of casein hydrolysate, and 0.8% agar (Sigma-Aldrich, St Louis, USA), with pH adjusted to 5.8) under a 16 h photoperiod using white fluorescent lamps (65 μmol/m2/s) at 22 °C (Wilson C R et al., 2010, Phytopathology, 100(5): 460-467). Individual seedlings of around 3 cm height with 3-4 fully expanded leaves were uprooted after 3 weeks and rinsed three times (10 mins each) in sterile distilled water to remove any adhering media. Plantlet roots were dipped into a broth culture of RR15 (Pantoea sp. RR15-treated root) or sterile distilled water (non-treated root) and left to immerse at room temperature for 5 h. The seedlings were transplanted into pots (17 cm×19 cm, with drainage holes in the bottom) filled with 2.5 L of field (ferrosol) and potting mix soil (1:1:8 mixes of peat, coarse sand, and composted pine bark) half of which had 500 mg of powdered S. subterranea (Sss) inoculum evenly distributed throughout the soil (+Sss) while the other half were uninoculated (Sss free) and then placed in a glasshouse environment with temperature maintained at 16 to 22±2 °C and a 16 h light/8 h dark photoperiod. Seedlings were irrigated every second day to maintain moist soil conditions. The experiment was split into two groups, one for each variety that was either treated or untreated with Pantoea sp. strain RR15. Each group included four treatments: (1) control (no RR15 or Sss); (2) RR15-treated and Sss-free; (3) RR15-treated with Sss; (4) No RR15 with Sss; each sample contained four biological replicates, each with one pot containing a single plant; samples were harvested destructively at three-time intervals following bacterial treatment: 4, 12 and 21 weeks for exudate analysis, bacterial recovery, disease assessment and plant growth parameters.

Recovery of Strain Pantoea sp. RR15

At each sampling period, the roots and soil were separated before further processing. Soil debris was removed from roots by shaking vigorously. The rhizosphere soil was then obtained from the root by placing the entire root in a 50 mL plastic conical tube containing 25 mL 0.1 M sterile phosphate buffer (7.1 g Na₂HPO₄, 4.4 g NaH₂PO₄H₂O added to 25 ml deionized water, pH 7.0), the tube was then centrifuged at 13000 g for 5 min, the roots were removed and the resulting soil suspension was centrifuged again at 4000×g for 15 min and the resulting soil pellet was regarded as the rhizosphere soil and kept at −20 °C until genomic DNA extraction (Liu H et al., 2021, New Phytol., 229(5): 2873-2885). Genomic DNA (gDNA) was extracted from 250 mg of rhizosphere soil fractions using the dNeasy Power Soil Pro Kit (Qiagen, USA) as per the manufacturer's recommendations. Soil DNA concentrations were determined using a Qubit® 2.0 Fluorometer (Life technologies, Darmstadt, Germany). The DNA samples were stored at −20 °C until further evaluation. Quantification of bacterial inoculant in the rhizosphere was performed using qPCR amplification of designed P1/P2 specific primers RR15-2f (5′-ACGGCCGCAAGGTTAAA-3′) (SEQ ID NO: 3) and RR15-2r (5′-GCTGGATGTCAAGAGTAGGTAAG-3′) (SEQ ID NO: 4), and probe RR15-2prb (5′-ACAAGCGGTGGAGCATGTGGTTTA-3′) (SEQ ID NO: 5), in a total reaction volume of 20 μL containing 2 μL template DNA, 0.5 μL of each 10 mM primer and probe, 10 μL of 1× SensiFAST™ Probe No-ROX Kit (Bioline Meridian BioScience, USA) and 5 μL sterile DNA-free water. The amplification was carried out with the Rotor-Gene Q system (Qiagen, Hilden, Germany) under the following thermocycling conditions: initial denaturation for one cycle at 95 °C for 30 secs, followed by amplification 40 times at 95 °C for 10 secs, 59 °C for 20 secs, and 72 °C for 20 secs. Each sample received three biological and technical replicates, and the marine bacteria Pseudoaltemoranas prydzensis was used as an internal reference to normalise the qPCR data for accurate quantification (Bowman J P, 1998, supra). A standard curve was developed using 10-fold serial dilutions ranging from 10⁸-10² copies per reaction of a gBlock fragment containing the targeted DNA PCR fragment of the bacterial 16S rRNA gene. The qPCR runs were calculated at 99% efficiency, with R2 value above 0.99%. The calibration curve was used to calculate the copy number of the target gene, and the Rotor-Gene Q software programme was used for all data processing (Qiagen, Hilden, Germany).

### Sampling Exudates and UPLC-MS/MS

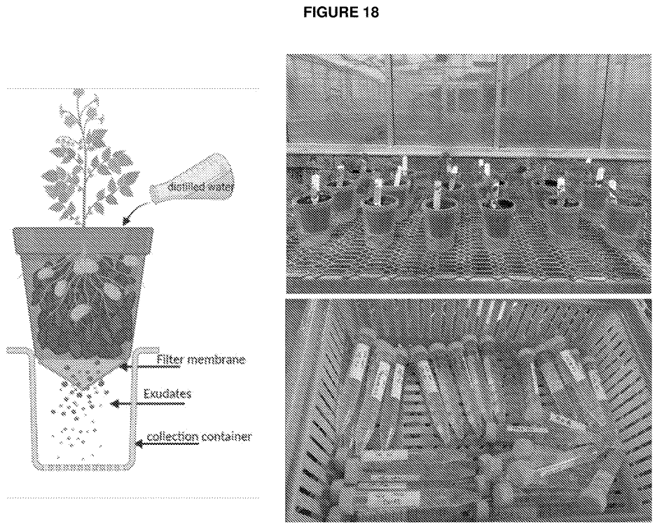

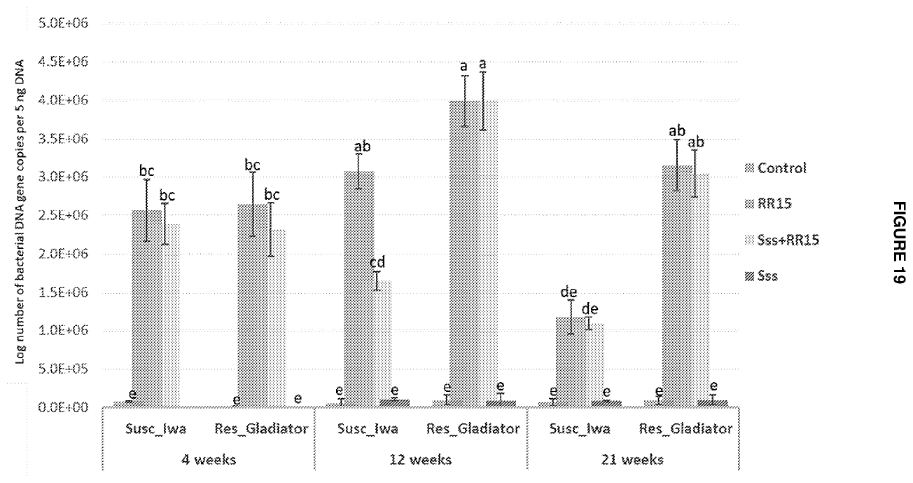

Samples of root exudate were obtained from each of the 72 potato plants in the pot study. Exudates were collected briefly by slowly pouring sterile water over the whole soil surface of the plant-soil system until 100 mL of exudates was collected from beneath the pot (FIG. 18). The collected exudates were centrifuged for 5 mins at 7000×g in a 50 mL falcon tube. Supernatant was collected and syringe filtered using a 0.22 μm filter (Merck Millipore), after which all samples were immediately frozen at −20 °C for storage (Williams A et al., 2021, supra). Exudate analysis was performed on thawed frozen root exudates, with 1 mL of each placed into HPLC glass vials. Root exudate extract was quantified using an ultra-performance liquid chromatography-tandem mass spectrometry (UPLC-MS/MS).

Potato metabolites were examined using a Waters Acquity H-Class UPLC system (Waters, Millford, MA) over a concentration range of 20 to 500 ug/mL. Chromatography was carried out using two UPLC columns that were connected in series: an Acquity BEH C18 column (2.1×100 mm×1.7 mm) and an Acquity HSS PFP column (2.1×100 mm×1.7 mm). Solvent A (1.0% v/v Acetic acid) and solvent B (Acetonitrile) were utilised in the mobile phase. The gradient program began with a 2-minute isocratic step at 100% mobile phase A, then proceeded to 70% mobile phase B over 4 minutes. 70% mobile phase B was held for 4 minutes before progressing to 90% mobile phase B over 1 minute and holding for 2 minutes. After that, the conditions were reset to original proportions and re-equilibrated for 3 minutes. The injection apparatus was subjected to two cycles of weak and strong solvent cleaning in between injections. The injection volume was 10 μL.

Multiple reaction monitoring (MRM) and an electrospray ionisation source in both positive and negative ion modes were used to identify metabolites. The electrospray parameters were as follows: capillary voltage of 2.5 kV, cone and desolvation temperatures of 150 °C and 400 °C respectively, with a desolvation gas flow of 950 L/h and a cone flow of 100 L/h. Standard compounds were used to optimise the cone voltage and collision energy for each analyte. The detection of expected [M+H]+ or [M H] molecules and product ions at the recognised retention indices confirmed the identification of metabolites. For each analyte, the MRM transition responses were averaged, and the MassLynx™ software was used to identify and quantify the peaks (Waters Corporation, Milford, MA).

The analysis of matrix-matched external calibration standards spanning the concentration ranges of 20 to 5000 ug/mL for organic compounds and 20 to 400 ug/L for amino acids was used for quantitation. External calibration standards were adjusted to account for natural analyte concentrations in the matrix. Separate analyses and quantitative measurements of glutamine and tyramine were performed using the same UPLC-MS/MS configuration but a different UPLC gradient algorithm. The gradient program started with a 1-minute isocratic step at 100% A, then advanced to 70% mobile phase B over 4 minutes and then held for 2 minutes. Afterwards, the conditions were reset to their original proportions and re-equilibrated for 3 minutes. Similarly, glutamine and tyramine were quantified by averaging the MRM transition responses and using the MassLynx™ software to locate and measure the peaks.

### Determination of Biomass Parameters and Root Disease

To measure yield-related characteristics, twelve sample plants were randomly harvested at full maturity from RR15-treated and untreated pots. Harvesting was carried out at 4, 12, and 21-weeks following inoculation. Root and shoot lengths (cm) and fresh weights (g) were measured, and tuber yield (g) was determined. Each plant was rated for root galling in which roots were washed and assessed using a visual scoring scale ranging from low to high corresponding the score of 0 to 4 where 0=no galls, 1=1-2 galls; 2=3-10 galls (<2 mm diameter); 3=≥10 galls (>2 mm diameter); 4=major roots with galls (>4 mm diameter) van de Graaf P et al., 2007, Plant Pathology, 56(6): 1005-1013). Chlorophyll content was estimated using a SPAD-502 (Konica Minolta Sensing) portable chlorophyll meter. SPAD measurements were performed in the centre of the lamina of the second topmost fully developed leaves. For each replicate, five readings per leaf were averaged to produce a single observation value (Kiana-Pouya A and Rasouli F, 2014, Photosynthetica, 52(2): 288-300).

### Statistical Analysis

Morphological traits and bacterial recovery were statistically analysed by using univariate analysis of variance (ANOVA) as two-tailed complete randomized blocks to compare the non-inoculated control plants vs. RR15-treated plants at different sampling periods. ANOVA was performed by the Tukey post-hoc test, with differences between means considered significant at p<0.05. Comparisons of metabolite abundances were carried out using the R package MetaboAnalystR version 3.0 (Xia J et al., 2009, Nucleic Acids Res., 37: web server issue W652-660), and the data were standardised using log transformation and then mean-centered. A fold change (FC) analysis supervised with t-test (p<0.05) plotted in log 2 scale was employed to compare absolute value changes of up/down regulated metabolites between groups and heatmaps were used to visualize differences of metabolite abundance among treatments and their biological replicates.

### Results

### Pantoea RR15 Enriched in the Root Environment in the Presence or Absence of Pathogen

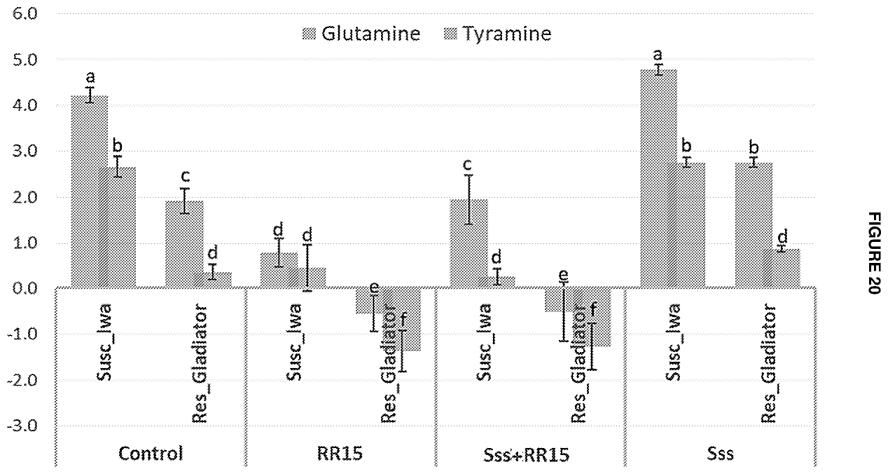

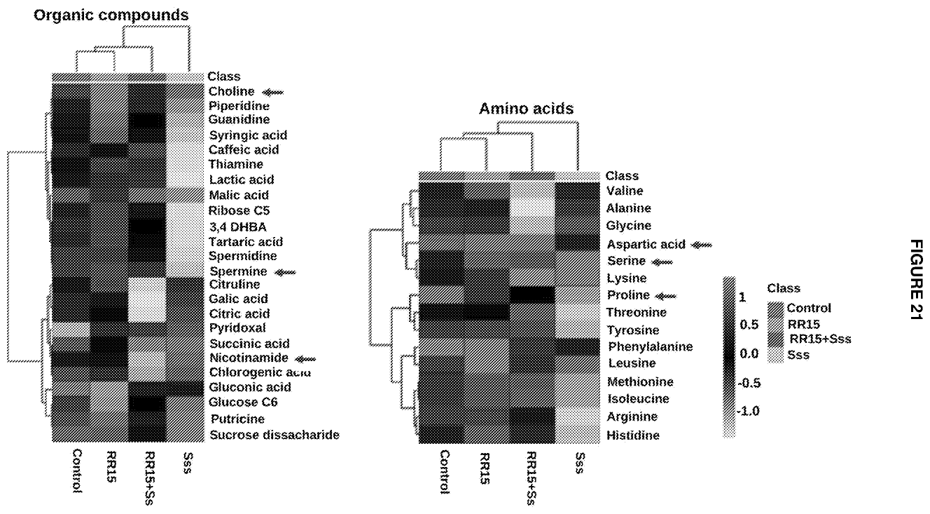

The abundance of Pantoea RR15 in soil was detected and evaluated using quantitative polymerase chain reaction (qPCR) (FIG. 19). Our findings demonstrated that RR15 was abundant around potato roots (up to 3.99×10⁶ log₁₀ DNA copies/5 ng total DNA) and was also detectable in roots of Sss-infected plants (up to 2.9×10⁶ log₁₀ DNA copies/5 ng total DNA). When bacteria were present alone or in combination with Sss, accumulation of Pantoea RR15 in the rhizosphere was significantly (p<0.05) different between susceptible (lwa) and resistant (Gladiator) potato cultivars as seen in later stages of crop growth (12-21 weeks). Notably, in the resistant cultivar, Pantoea RR15 was observed to steadily increase in density at the onset of four weeks (2.65×10⁶ log₁₀ DNA copies/5 ng total DNA) and progressed until 12 weeks (4×10⁶ log₁₀ DNA copies/5 ng total DNA) before gradually reducing to 3.15×10⁶ log₁₀ DNA copies/5 ng total DNA after 21 weeks. Meanwhile, in the presence of Sss, RR15 populations declined moderately, with DNA levels from 2.32×10⁶ log₁₀ DNA copies/5 ng total DNA at 4 weeks and continually dropping to 1.65×10⁶ log₁₀ DNA copies/5 ng total DNA and 1.09×10⁶ log₁₀ DNA copies/5 ng total DNA after 12 and 21 weeks, respectively.

On the other hand, when Pantoea RR15 was delivered alone in the rhizosphere to the susceptible cultivar, the abundance of Pantoea RR15 was relatively high at the start of 4 weeks (up to 2.56×10⁶ log₁₀ DNA copies/5 ng total DNA) and continually increased until 12 weeks with a DNA level of 3.07×10⁶ log₁₀ DNA copies/5 ng total DNA, but then declined sharply to 1.17×10⁶ log₁₀ DNA copies/5 ng total DNA after 21 weeks. Similar downward trends were observed when Sss were present, with 2.39×10⁶ log₁₀ DNA copies/5 ng total DNA at the beginning of 4 weeks, then proceeding to decline until nearly 1.09×10⁶ log₁₀ DNA copies/5 ng total DNA were detected after 21 weeks. Overall, it can be assumed that Pantoea RR15 reached the maximum carrying capacity for rhizosphere establishment at approximately 12 weeks, then gradually declined thereafter. Furthermore, a substantial difference in bacterial recruitment was identified between cultivars, with Pantoea RR15 colonising more effectively in the resistant variety rather than the susceptible one.

### Sss-Stimulating Compound Degradation by Pantoea RR15 in the Rhizosphere

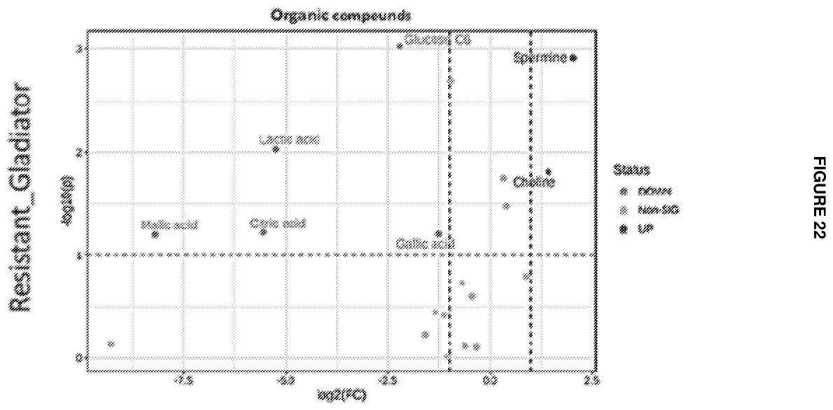

To assess degradation of Sss-stimulants (glutamine and tyramine) by microbe-plants interactions, Pantoea RR15 was introduced onto the roots of potato plants with bacteria given alone or in addition with pathogen (Sss) (FIG. 20). Notably, the levels of glutamine and tyramine in potato exudates were consistently different. In contrast to tyramine, a higher quantity of glutamine was released. Glutamine and tyramine were generally present in greater quantities in the susceptible cultivar, with average estimated concentrations of 2.9±0.8 mg/mL and 1.5±2.6 mg/mL, respectively, whereas the resistant cultivar showed negligible levels, with glutamine averaging only about 0.9±2.8 mg/mL and tyramine of 0.3±0.9 mg/mL. Inoculation with RR15 alone dramatically reduced the quantity of glutamine and tyramine in the rhizosphere, as evidenced in both susceptible and resistant cultivar exudates. A similar response was observed when RR15 was inoculated at the same time as the pathogen. Moreover, the level of glutamine and tyramine from the resistant cultivar was below the detection limit, whereas from the susceptible cultivar, the decline of glutamine and tyramine varied. RR15-treated plants had a glutamine reduction of up to 0.8 mg/mL and a tyramine reduction of up to 0.4 mg/mL, whereas RR15 with pathogen present had glutamine and tyramine reductions of 1.9 mg/ml and 0.2 mg/mL, respectively. Plant root exudates without bacterial treatment had a comparable amounts of glutamine (1.9 to 4.2 mg/mL) and tyramine (0.4 to 2.7 mg/mL) levels, while pathogen-infected plants had substantially higher glutamine (2.8 to 4.8 mg/mL) and tyramine (0.9 to 2.8 mg/mL) levels.

### Pantoea RR15 Shaping Secondary Metabolite Composition

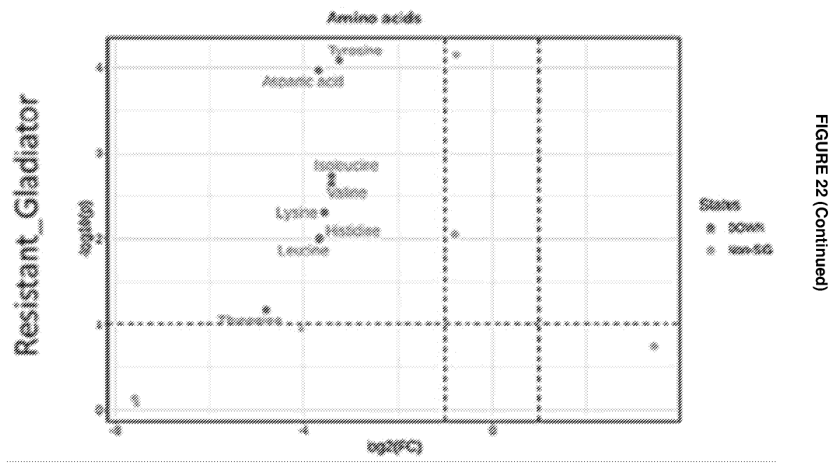

To explore the potential role and influence of Pantoea RR15 on potato root exudation, an established MRM-based targeted metabolomics technique was employed. The results revealed that the level of certain metabolites changed significantly between treated and untreated plants. The metabolic makeup of RR15-treated plants revealed highly variable fluctuations of the root exudate metabolome, including when RR15 was inoculated with the pathogen, as illustrated in heatmap clustering of metabolites (FIG. 21).

It was found that amino acids (proline, serine, and aspartic acid) and organic compounds (nicotinamide) that are reportedly linked to Sss activation were abundant in non-treated plant exudate and were reduced both in RR15-treated plants exudates and even when RR15 was introduced with Sss. Conversely spermine and choline, which are reportedly inhibitors of Sss zoospore chemotaxis, were enhanced with RR15 application. Intriguingly, plants exposed to Sss alone, showed changes of amino acids and organic compounds that were distinct from the control of those that were treated with RR15. It is clear that low molecular weight organic compounds (LMWO), presumed to be stimulatory for Sss zoospore release, such as aspartic acid, serine, nicotinamide, citrulline and citric acid, were highly abundant in Sss infected potatoes, whereas non-stimulant compounds such as choline and spermine were undetectable. Notably, when RR15 was introduced, the overall changes in stimulatory and non-stimulatory substances were very apparent (FIG. 21).

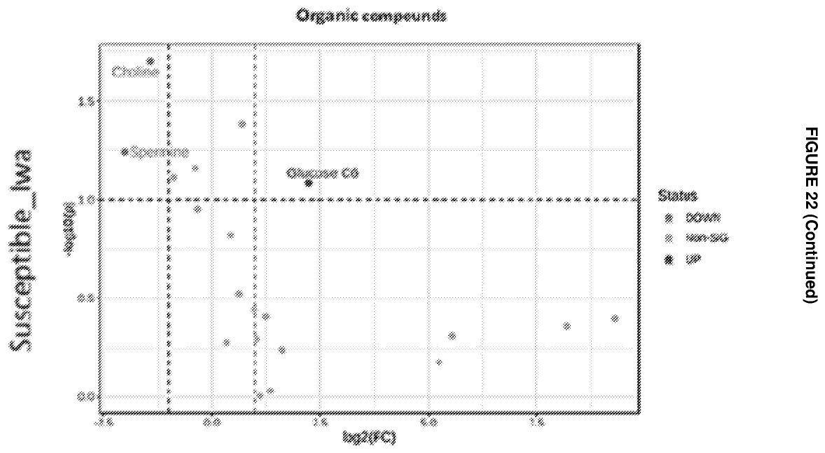

As shown in FIG. 22, the metabolic components of potato root exudates varied between resistant and susceptible cultivars. The organic compounds, lactic acid (log 2 FC=−5.2), malic acid (log 2 FC=−8.2), citric acid (log 2 FC=−5.5), gallic acid (log 2 FC=−1.2), and glucose C6 (log 2 FC=−2.2), were downregulated in the resistant cultivar, as were the amino acids, tyrosine (log 2 FC=−3.2), aspartic acid (log 2 FC=−3.4), isoleucine (log 2 FC=−3.7), valine (log 2 FC=−3.4), lysine (log 2 FC=−3.6), histidine (log 2 FC=−3.7), leucine (log 2 FC=−3.6), and threonine (log 2 FC=−4.8). The susceptible cultivar, on the other hand, exhibited fewer metabolite changes with amino acids, histidine (log 2 FC=−3.6), methionine (log 2 FC=−3.52) and arginine (log 2 FC=−4.8), decreasing, while leucine (log 2 FC=2.1), phenylalanine (log 2 FC=1.2) and glucose C6 (log 2 FC=1.8) increased. Remarkably, the major discriminating metabolites identified between resistant and susceptible cultivars revealed non-stimulatory metabolites spermine (log 2 FC=2.98) and choline (log 2 FC=1.62) being accumulated in larger amounts compared to susceptible cultivars, which had measured levels of log 2 FC=−2.2 and log 2 FC=−1.4, respectively.

### Pantoea RR15 Promotes Plant Development

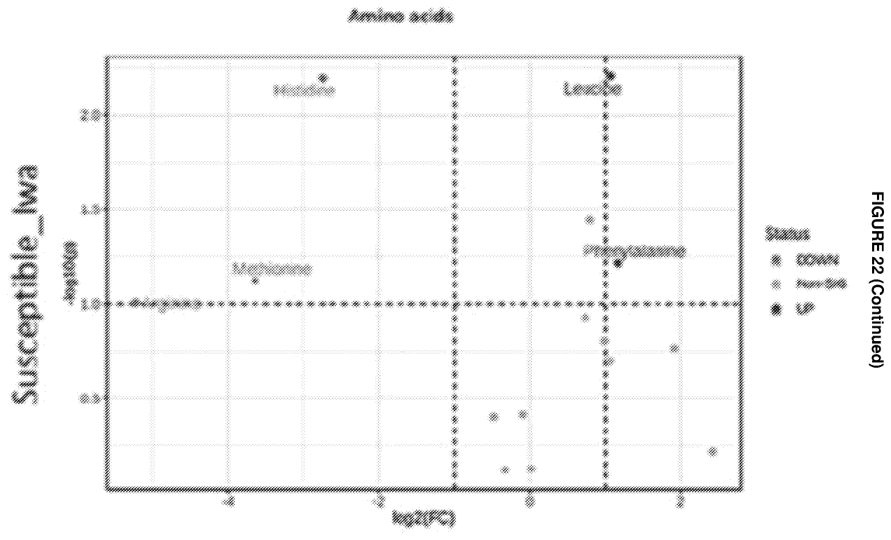

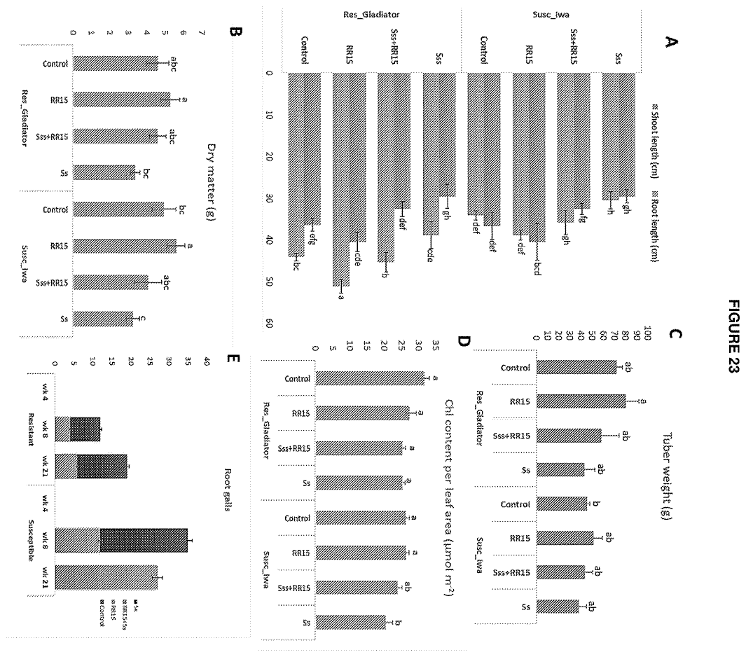

In glasshouse conditions, the effect of bacterial strain Pantoea RR15 on growth promotion and its biocontrol action against Sss were assessed (FIG. 23). At 21 weeks after treatment, the results show that inoculating potatoes with the strain Pantoea RR15 was the most effective in boosting growth, with a significant (p<0.05) influence on enhancement of the root system. In disease-free plants treated with RR15, root (ave. 40.5-51.2 cm/plant) and shoot (ave. 38.9-40.5 cm/plant) increased significantly by 13% and 11.5%, respectively. Infected plants treated with RR15 showed a 0.7% increase in root (ave. 35.9-45.4 cm/plant) and 10.2% increase in shoot (ave. 34.2-44.2 cm/plant) (FIG. 23A). Remarkably, the growth promotion impact of Pantoea RR15 on root and shoot development was more pronounced in disease-free plants, however this effect was also seen in Sss-infected plants treated with RR15. In addition, RR15-treated plants had a 17.8% total dry weight increase over non-treated controls and a 31.4% total dry weight increase over pathogen alone treatment; however, no significant differences in dry mass accumulation were observed between Sss-infected plants treated with RR15 and non-treated controls. (FIG. 23B).

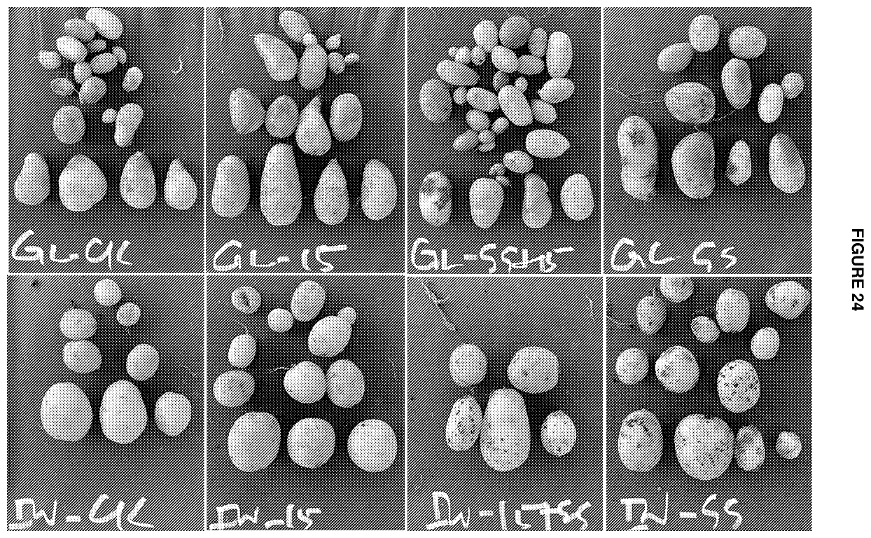

Tuber development screening demonstrated that RR15-treatment, particularly in resistant cultivars, increased tuber fresh mass yield while having minimal effect on susceptible varieties (FIG. 24). Fresh tuber weights from RR15-treated resistant potatoes averaged 80.6 g/plant, compared to 51.1 g/plant in RR15-treated susceptible potatoes, although tubers generated from bacterially treated plants were greater than non-treated controls, with average tubers weights of 71.8 g/plant in resistant plants and 45.5 g/plant in susceptible plants (FIG. 23C).

Chlorophyll content of healthy plants, regardless of whether resistant (36.7 μmol m⁻² per leaf area) or susceptible (31.8 μmol m⁻² per leaf area), had higher chlorophyll content than infected resistant (25.3 μmol m⁻² per leaf area) and the susceptible cultivar (20.5 μmol m⁻² per leaf area) (FIG. 23D). Furthermore, results revealed that total chlorophyll content was constantly higher in the resistant cultivar compared to the susceptible cultivar, and that whether bacterially treated or not, results indicated a higher estimate of chlorophyll production in the resistant cultivar than the susceptible one even with RR15 inoculation (Res_gladiator=27.4 μmol m² per leaf area vs. Sus_lwa=26 μmol m⁻² per leaf area) or with RR15 in combination with pathogen (Res_gladiator=25.3 μmol m⁻² per leaf area vs. Sus_lwa 23.9 μmol m⁻² per leaf area).

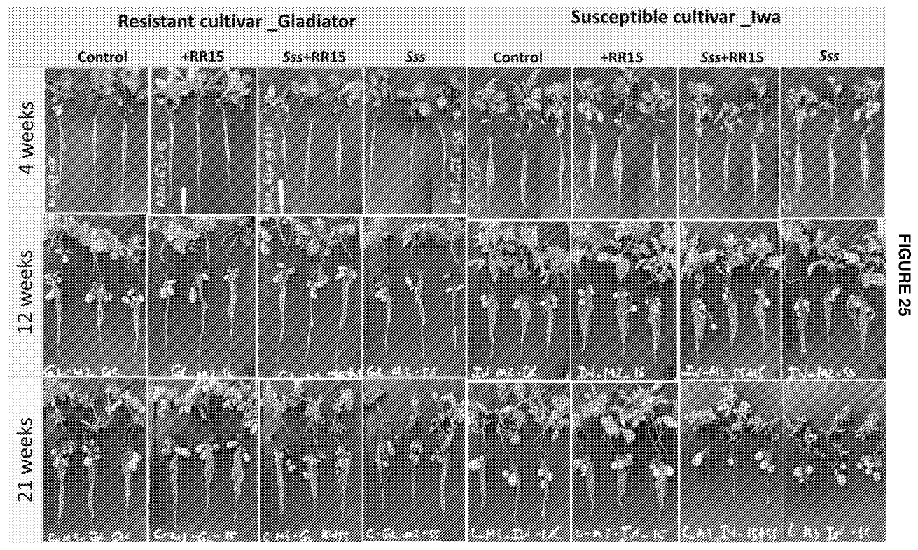

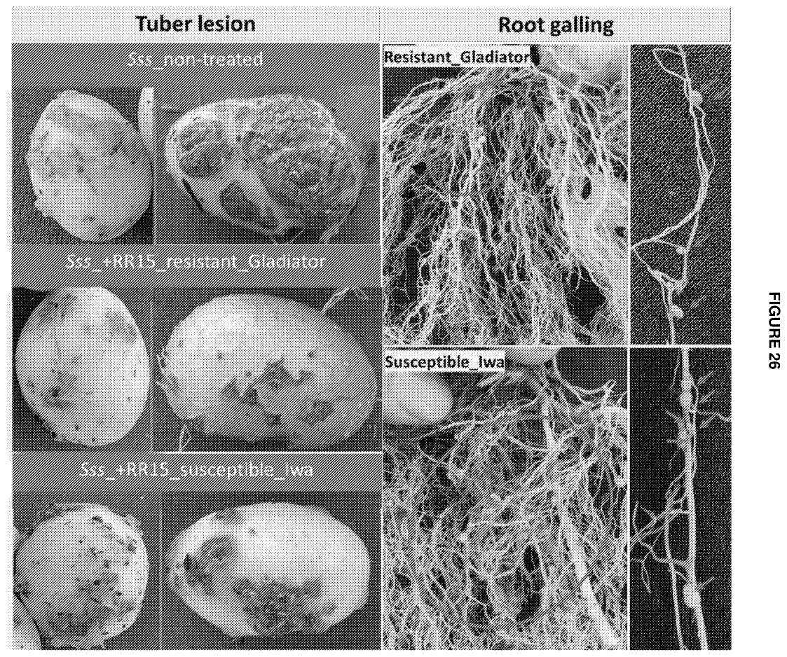

Additionally, the biocontrol activity of Pantoea RR15 was detectable. No disease symptoms were detected in RR15-treated plants in the entire pot experiment. The most serious infections were found in plants that were exclusively supplied with Sss. However, plants infected with Sss and provided with RR15 developed more roots (32.6-35.9 cm/plant) and shoots (32.6-35.9 cm/plant) than when the pathogen was applied alone (shoot=29.6-30.5 cm/plant; root=28.9-38.9 cm/plant) (FIG. 23A). Furthermore, RR15 pre-treatment lowered root galling, indicating that it may have a role in infection prevention, since the number of galls assessed in infected potato seedlings inoculated with RR15 was lower than in infected potato seedlings not treated with RR15 (FIG. 23E). It is worth emphasising that the detection of disease symptoms such as tuber lesions and root rotting is clearly extensive in infected plants in the absence of bacterial treatment. Disease symptoms are obviously more evident in susceptible cultivars than in resistant cultivars; however, lower root rot degeneration in infected resistant cultivar indicated that Pantoea RR15 played an essential role in mitigating disease impacts (FIGS. 25 and 26).

### Discussion

Metabolomics is gaining importance as a tool for efficiently distinguishing metabolite origins and studying the roles of plant-microbe interactions. Because of the input of exogenous compounds from all community members, the presence of a phytopathogen, beneficial microorganism, and host plant is likely to create changes in each of their distinct metabolomes. The establishment of introduced bacteria on roots, which is a vital stage in the formation of plant-microbe interactions was examined in this study. Furthermore, metabolomics analysis was performed to evaluate the change in metabolic state of potato exudation as a result of bacterial colonisation in the rhizosphere, and to determine if it improves plant viability and growth while also protecting the potato invading phytopathogens.

Our findings revealed that 21 weeks after inoculation, the efficiency of colonisation of strain Pantoea RR15 in the root environment is consistent and robust across both resistant and susceptible cultivars. Pantoea RR15 growth dynamics in the rhizosphere of both cultivars were constant resulting in a stable population with DNA level of 2.6×10⁶-4×10⁶ log₁₀ DNA copies/5 ng total DNA in the potato root habitat even when the pathogen was present (1×10⁶-3×10⁶ log₁₀ DNA copies/5 ng total DNA). Additionally, the gradual decrease in bacterial population over time observed in the enumeration of Pantoea RR15 evaluated from resistant (from 4×10⁶ to 3.2×10⁶ log₁₀ DNA copies/5 ng total DNA) and susceptible (from 3.1×10⁶ to 1.2×10⁶ log₁₀ DNA copies/5 ng total DNA) cultivars confirms similar observations to the colonisation pattern of strain Pantoea SWg2 reported by Xie J et al., 2017, Biological Control, 113:9-17, suggesting that enumeration of SWg2 in roots increased up to 10⁸ CFU g⁻¹ in 3 DPI then declined slowly to 10³ CFU g⁻¹ in 28 DPI. The successful establishment of Pantoea RR15 in the potato root habitat, as depicted in this study, may have resulted in motility characteristics that potentially contributed to the fast attachment process, as previously observed by Wheatley RM and Poole P S, 2018, FEMS Microbiol. Rev., 42(4): 448-461, who confirmed that the Pantoea root attachment process is driven by pili and attachment proteins (as in plant-colonizing pseudomonads, likely in conjunction with or in response to chemical cues from the plant). Similarly, in Pantoea sp. The YR343 isolate (Bible A N et al., 2016, Frontiers in Microbiol., 7: Article 491) has been shown to have a variety of features that improve their capacity to survive and established in the rhizosphere, including swimming and swarming motility.

The efficacy of Pantoea RR15 treatment to promote plant development in both resistant and susceptible cultivars as shown in this study was validated by visible root improvement and a considerable increase in plant biomass. The extent of the plant growth promoting effects of Pantoea RR15 reported in this study is consistent with the fact that Pantoea is thought to harbour a diverse repertoire of plant growth-promoting traits, including indole-3-acetic acid production and phosphorus solubilization. Phosphorus is one of the most essential mineral elements for seedling growth, yet it is frequently contained in soil in insoluble forms, such as calcium phosphates, rendering it unavailable to plants (Rodriguez H et al., 2006, Plant and Soil, 287(1-2): 15-21). Some plant growth-promoting microorganisms, as evidenced in most Pantoea isolates, can dissolve mineral phosphates and facilitate phosphate uptake by plants, encouraging plant development (Zaidi S et al., 2006, Chemosphere, 64:991-997).

### Conclusion

Recent advances in metabolomics were employed to investigate the changes in root exudation patterns imposed by plant treatment with Pantoea RR15. Overall, the metabolic profile in potato was diverse and was altered in a completely different manner following RR15 inoculation. Given the fact that the mechanisms behind changes in these below-ground chemical signals are yet unclear, our findings suggest that microbial inoculation may alter the exudate profile in both healthy and diseased plants. To the best of our knowledge, no information on the metabolic reactions of microbial inoculation with potato and powdery scab is currently available. These findings contribute to our knowledge of the role of Pantoea RR15 in supporting various biostimulant activities, particularly in plant growth regulation. As a result, we therefore speculate that one of the processes behind the enhancement of plant growth mediated by Pantoea RR15 is the regulation of root exudate composition. From a practical standpoint, our findings might be useful in the development of innovative ways for addressing crop tolerance to soil-borne disease infection.

### Example 4

### Field Testing of Bacterial Inoculant for Plant Growth Promotion and Yield Enhancement

### Materials and Methods

### Production of Inoculant

Inoculant of the bacterial strain (RR15) was prepared 1 week prior to use. The bacterial strain RR15 was cultured in 200 mL LB broth at 25 °C overnight in a 2 L flask on an orbital shaker. The bacterial culture was centrifuged (3000 rpm) to concentrate cells and 100 mL of concentrated bacterial suspension was added to 50 mL of sterile 15% glycerol and 50 mL sterile 1% Xanthan gum. 5 g of 2% bacteriological peptone was added. The bacterial mixture was then combined with sterilised vermiculite (250 g) and spread in a thin layer on a sterile tray in a laminar flow cabinet overnight to air-dry. The air-dried inoculant was stored in a polypropylene bag until used.

### Seed Tuber Preparation and Trial Establishment

Certified (disease-free) seed tubers of cv. Russet Burbank were used. Approximately 100 cut setts (approximately 50 g each) of seed tubers were coated with the inoculant. For the control 100 setts remained untreated. Seed tubers were planted in a randomised block design in a field site at Forthside Vegetable research facility (Tasmania, Australia). Treated tubers and control tubers were planted in five replicated plots each containing 2 rows of 10 tubers. The soil type was a red ferrosol typical of the region. Fertiliser and irrigation practices used during the trial were typical of commercial use.

### Trial Harvest and Scoring

Plots were harvested approximately 6 months after planting. All tubers from five plants per plot were harvested. Tubers were counted and individually weighed. The distribution of tubers within commercially acceptable size and weight ranges was calculated.

### Results

Emergence of potato plants from treated plots occurred earlier than from control plots, and plants appeared stronger and more vigorous early in their growth. Differences in foliage development were less obvious as the plants matured. At harvest no disease was noted on any tubers from control or treated plots. Tubers from treated plots were clearly visibly larger on average. Assessment of tuber numbers and weight are summarised in Table 4 below.

TABLE 4

| | | | Mean | Mean | | Mean | |
|---|---|---|---|---|---|---|---|
| | | Mean | Ave | Mkt | Mean | Ideal | Mean |
| | Mean | Total | wt | wt | Mkt | wt | Ideal |
| | tuber | wt | (g)/ | (>75 | wt | (250- | wt |
| | count | (g) | tuber | g) | count | 850) | count |
| Treated | 57.6 | 9920.4 | 175.0 | 9428.6 | 43.5 | 3772.0 | 11.3 |
| Control | 67.4 | 8691.3 | 132.3 | 7849.3 | 46.4 | 2250.5 | 7.2 |

The mean count indicated control plots had on average greater tubers numbers than treated plots. The average weight per tuber was greater in treated plots. Total tuber weight was greater in the treated plots (9.9 kg) than control plots (8.7 kg) and appears consistent (across reps) with an average 12.3% increase. Marketable tuber weight (which excludes all chats <75 g) shows tuber numbers are equivalent between control and treatment, with tuber yields greater in treated plots with an average 16.8% increase. The Ideal weight is in the size class 250-850 g. Treated plots (3.8 kg) had greater numbers and yield in this class than control plots (2.3 kg) with an average 40.3% increase.

### Conclusions

Potato seed tubers treated with bacterial inoculant prepared in a vermiculite carrier with Gycerol and Xanthan gum as bacterial stabilisers showed enhanced emergence and early growth which was reflected in greater tuber yields. Notably tubers from treated plots were on average larger and more fell within marketable and ideal size weight categories.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be noted that where a range of values is expressed, it will be clearly understood that this range encompasses the upper and lower limits of the range, and all numerical values or sub-ranges in between these limits as if each numerical value and sub-range is explicitly recited. The statement “about X % to Y %” has the same meaning as “about X % to about Y %,” unless indicated otherwise.

The term “about” as used in the specification means approximately or nearly and in the context of a numerical value or range set forth herein is meant to encompass variations of +/−10% or less, +/−5% or less, +/−1% or less, or +/−0.1% or less of and from the numerical value or range recited or claimed.

It is also to be noted that, as used herein, the singular forms “a”, “an” and “the” include plural aspects unless the context already dictates otherwise.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Finally, reference is made to standard textbooks of molecular biology that contain methods for carrying out basic techniques encompassed by the present invention. See, for example, Green M R and Sambrook J, Molecular Cloning: A Laboratory Manual (4th edition), Cold Spring Harbor Laboratory Press, 2012.

1. An isolated bacterial strain RR15 of the genus Pantoea, deposited under Accession Number V22/011015 on 9 Jun. 2022 with the National Measurement Institute, Australia.

2. A culture of the bacterial strain of claim 1, or a supernatant obtained from said culture.

3-8. (canceled)

9. The bacterial strain of claim 1, wherein the plant is of the Solanaceae family, or wherein the plant is of the species Solanum tuberosum.

10-17. (canceled)

18. A method for promoting plant growth, the method comprising applying to the plant or to soil in which the plant will be, or is, propagated in, an effective amount of the bacterial strain.

19. A method for promoting plant tuber yield, the method comprising applying to the plant or to soil in which the plant will be, or is, propagated in, an effective amount of the bacterial strain of claim 1.

20. The method of claim 19, wherein tuber size and/or weight is promoted.

21. A method for inhibiting plant root infection by a pathogen, the method comprising applying to the plant or to soil in which the plant will be, or is, propagated in, an effective amount of the bacterial strain of claim 1.

22. A method for inhibiting plant root disease by a pathogen, the method comprising applying to the plant or to soil in which the plant will be, or is, propagated in, an effective amount of the bacterial strain of claim 1.

23. (canceled)

24. A composition comprising a supernatant and/or culture of an isolated bacterial strain RR15 of the genus Pantoea, of claim 1 deposited under Accession Number V22/011015 on 9 Jun. 2022 with the National Measurement Institute, Australia.

25. (canceled)

26. The composition comprising the bacterial strain of claim 1, wherein the composition promotes plant growth.

27. The composition comprising the bacterial strain of claim 1, wherein the composition promotes plant root growth.

28. The composition comprising the bacterial strain of claim 1, wherein the composition promotes plant tuber yield and wherein tuber size and/or weight is promoted.

29. (canceled)

30. The composition of claim 26, wherein the plant is of the Solanaceae family or wherein the plant is of the species Solanum tuberosum.

31. (canceled)

32. The bacterial strain of claim 1, which inhibits plant root infection by a pathogen, wherein the pathogen is Spongospora subterranean.

33. The bacterial strain of claim 1, which inhibits plant root disease by a pathogen, wherein the pathogen is Spongospora subterranean.

34. (canceled)

35. The composition of claim 22, further comprising a biological agent, which is a plant growth promoting agent.

36. (canceled)

37. The composition of claim 22, further comprising a bacterial stabilising agent, which is xanthan gum.

38-41. (canceled)

42. A method for promoting plant tuber yield or inhibiting plant root infection by a pathogen, the method comprising applying to the plant or to soil in which the plant will be, or is, propagated in, an effective amount of:
(i) an isolated bacterial strain RR15 of the genus Pantoea, deposited under Accession Number V22/011015 on 9 Jun. 2022 with the National Measurement Institute, Australia;
(ii) a culture of an isolated bacterial strain RR15 of the genus Pantoea, deposited under Accession Number V22/011015 on 9 Jun. 2022 with the National Measurement Institute, Australia; and/or
(iii) a supernatant of a culture of an isolated bacterial strain RR15 of the genus Pantoea, deposited under Accession Number V22/011015 on 9 Jun. 2022 with the National Measurement Institute, Australia; or
(iv) a composition comprising (i), (ii), and/or (iii).

43. The method of claim 42, wherein tuber size and/or weight is promoted.

44-51. (canceled)

52. A composition comprising:
(i) an isolated bacterial strain selected from one or more of the group consisting of RR15, RR09, RR08, RR17, RR12, RR01, RR04, RR14, RR07 and RR10;
(ii) a culture of an isolated bacterial strain selected from one or more of the group consisting of RR15, RR09, RR08, RR17, RR12, RR01, RR04, RR14, RR07 and RR10; and/or
(iii) a supernatant of a culture of an isolated bacterial strain selected from one or more of the group consisting of RR15, RR09, RR08, RR17, RR12, RR01, RR04, RR14, RR07 and RR10.

53-55. (canceled)
